(19) United States
(12) Patent Application Publication
(10) Pub. No.: US 2023/0108442 A1
(43) Pub. Date: Apr. 6, 2023

(54) NOVEL ANTIMALARIAL AGENT CONTAINING HETEROCYCLIC COMPOUND

(71) Applicant: Eisai R&D Management Co., Ltd., Tokyo (JP)

(72) Inventors: Takaaki HORII, Tsukuba-shi, Ibaraki (JP); Shuichi SUZUKI, Tsukuba-shi, Ibaraki (JP); Andrew James Amin ROUPANY, Hatfield, Hertfordshire (GB); James Jun Bon MUI, Hatfield, Hertfordshire (GB); Joseph Peter LEWIS-MARSHALL, Hatfield, Hertfordshire (GB); Francesca Ann DEVAN, Hatfield, Hertfordshire (GB); Matthew William CARTWRIGHT, Hatfield, Hertfordshire (GB)

(21) Appl. No.: 17/788,017

(22) PCT Filed: Jan. 19, 2021
(86) PCT No.: PCT/JP2021/001735

§ 371 Date: Jun. 22, 2022

Related U.S. Application Data
(60) Provisional application No. 63/085,511, filed on Sep. 30, 2020.
(60) Provisional application No. 62/963,714, filed on Jan. 21, 2020.

(51) Int. Cl. C07D 401/04; A61P 33/06

(52) U.S. Cl. CPC C07D 401/04; A61P 33/06

(57) ABSTRACT

Disclosed herein are diaminopyridine compounds or pharmaceutically acceptable salts thereof having antimalarial activation effect.

### TECHNICAL FIELD

The present invention relates to a novel antimalarial agent containing a heterocyclic compound.

### BACKGROUND ART

Malaria is a life-threating infectious disease caused by Plasmodium protozoa. An estimated 228 million people were infected and 405.000 people died in 2018, mainly children in African countries. Many countries, companies and scientists are actively cooperating to work to achieve the eradication of malaria. The current standard care for the treatment of malaria typically involves combination therapy with artemisinins. To date, unlike some other approved antimalarial drugs, artemisinins have a very favorable safety profile with few adverse events associated with their use. The problem is that artemisinin containing combinations have already been associated with resistance rates as high as 45% in places like Cambodia, Thailand, and Vietnam (See NPL 1 and 2). Given the high rate of resistance that has developed in a fairly short period of time, a new class of compounds with novel mechanism-of-action (MoA) is urgently required to treat resistant strains of malaria parasites and support the malaria eradication strategy.

Glycosylphosphatidylinositol (GPI) is a common moiety in all eukaryotes and has a role in anchoring many proteins to the cell surface. The biosynthetic pathway of GPI is well studied and as one of the essential enzymes, Gwt1p, was identified as a novel target for an antifungal drug (See NPL 3 to 6). Gwt1p catalyzes the acylation of inositol in the early part of the GPI biosynthesis pathway. In the course of biological studies, it was found that the GWT1 gene encoding Gwt1p enzyme is highly conserved among eukaryotes, including Plasmodium protozoa, the etiological pathogens for malaria. The preliminary hit compound with inhibitory activities on plasmodial Gwt1p showed anti-Plasmodium activities in vitro and in vivo. Therefore, a compound that inhibits selectively the biosynthesis of GPI, and in particular the acylation of the inositol ring, may be an extremely useful antimalarial agent.

PTL 1 is the prior art related to antimalarial agent based on such a mechanism. Described in PTL 1 are heterocyclic compounds having antimalarial activity by inhibition of the biosynthesis of GPI via inhibition of the activity of the GWT1 gene product from malaria protozoa. However, the compounds disclosed in PTL 1 have 2-benzyl pyridine as a common structure, and clearly differ structurally from the compound according to the present invention.

Meanwhile, PTL 2 is the prior arts that are most similar structurally to the heterocyclic compounds (1-IX) according to the present invention. PTL 2 discloses N-unsubstituted diamino pyridine derivatives. However, not only the compound according to the present invention, but 5-substituted diamino pyridine derivatives, are not at all disclosed in PTL 2.

### CITATION LIST

### Patent Literature

[PTL 1] WO 2004/048567
[PTL 2] WO 2006/016548

### Non Patent Literature

[NPL 1] Yeung S, Socheat D, Moorthy V S et al. Artemisinin resistance on the Thai-Cambodian border. Lancet 2009; 374: 1418-9.
[NPL 2] Hawkes M, Conroy A L, Kain K C. Spread of artemisinin resistance in malaria. The New England journal of medicine 2014; 371: 1944-5.
[NPL 3] Okamoto M, Yoko-o T, Umemura M et al. Glycosylphosphatidylinositol-anchored proteins are required for the transport of detergent-resistant microdomain-associated membrane proteins Tat2p and Fur4p. The Journal of biological chemistry 2006; 281: 4013-23.
[NPL 4] Sagane K, Umemura M, Ogawa-Mitsuhashi K et al. Analysis of membrane topology and identification of essential residues for the yeast endoplasmic reticulum inositol acyltransferase Gwt1p. The Journal of biological chemistry 2011; 286: 14649-58.
[NPL 5] Tsukahara K, Hata K, Nakamoto K et al. Medicinal genetics approach towards identifying the molecular target of a novel inhibitor of fungal cell wall assembly. Mol Microbiol 2003; 48: 1029-42.
[NPL 6] Umemura M, Okamoto M, Nakayama K et al. GWT1 gene is required for inositol acylation of glycosylphosphatidylinositol anchors in yeast. The Journal of biological chemistry 2003; 278: 23639-47.

### SUMMARY OF INVENTION

### Technical Problem

It is an object of the present invention to provide an excellent antimalarial agent.

### Solution to Problem

As a result of extensive studies to address the above problems, the present inventors found a diamino pyridine compound or a pharmaceutically acceptable salt thereof having antimalarial action.

Specifically, the invention relates to the following <1> to <17>.

<1> a compound selected from the group consisting of:
(R)-2,6-diamino-5-(3-(trifluoromethyl)-1H-1,2,4-triazol-1-yl)-N-(4-((1,1,1-trifluoropropan-2-yl)oxy)benzyl)nicotinamide represented by the following formula (I):

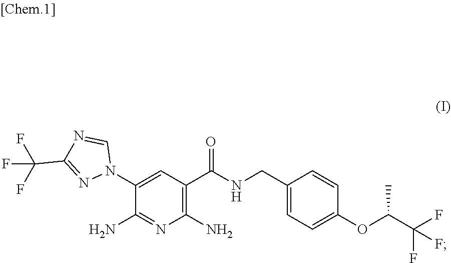 [SMILES: C[C@@H](Oc1ccc(CNC(=O)c2cc(-n3cnc(C(F)(F)F)n3)c(N)nc2N)cc1)C(F)(F)F]

(R)-2,6-diamino-5-(1-(difluoromethyl)-1H-pyrazol-4-yl)-N-(4-((1,1,1-trifluoropropan-2-yl)oxy)benzyl)nicotinamide represented by the following formula (II):

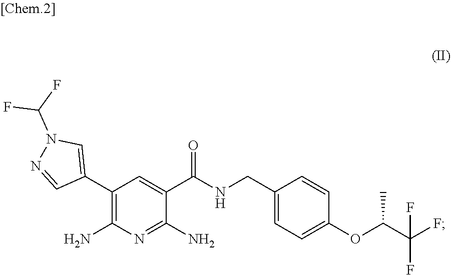 [SMILES: C[C@@H](Oc1ccc(CNC(=O)c2cc(-c3cnn(C(F)F)c3)c(N)nc2N)cc1)C(F)(F)F]

(R)-2,6-diamino-5-(1-(difluoromethyl)-3-methyl-1H-pyrazol-4-yl)-N-(4-((1,1,1-trifluoropropan-2-yl)oxy)benzyl)nicotinamide represented by the following formula (III):

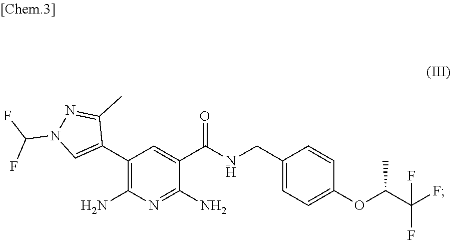 [SMILES: Cc1nn(C(F)F)cc1-c1cc(C(=O)NCc2ccc(O[C@H](C)C(F)(F)F)cc2)c(N)nc1N]

2,6-diamino-N-(4-(2,2,2-trifluoroethoxy)benzyl)-5-(3-(trifluoromethyl)-1H-1,2,4-triazol-1-yl)nicotinamide represented by the following formula (IV):

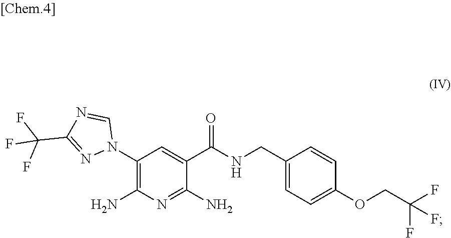 [SMILES: Nc1nc(N)c(-n2cnc(C(F)(F)F)n2)cc1C(=O)NCc1ccc(OCC(F)(F)F)cc1]

(R)-2,6-diamino-5-(3-(difluoromethyl)-1H-1,2,4-triazol-1-yl)-N-(4-((1,1,1-trifluoropropan-2-yl)oxy)benzyl)nicotinamide represented by the following formula (V):

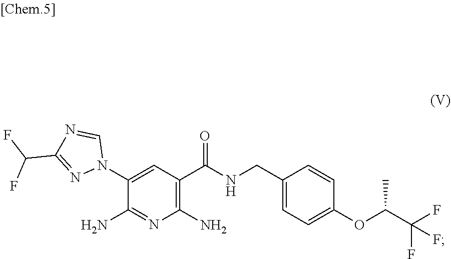 [SMILES: C[C@@H](Oc1ccc(CNC(=O)c2cc(-n3cnc(C(F)F)n3)c(N)nc2N)cc1)C(F)(F)F]

(R)-2,6-diamino-5-(3-(2,2,2-trifluoroethyl)-1H-1,2,4-triazol-1-yl)-N-(4-((1,1,1-trifluoropropan-2-yl)oxy)benzyl)nicotinamide represented by the following formula (VI):

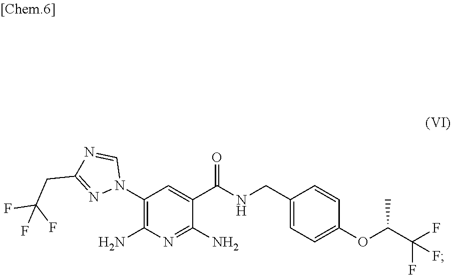 [SMILES: C[C@@H](Oc1ccc(CNC(=O)c2cc(-n3cnc(CC(F)(F)F)n3)c(N)nc2N)cc1)C(F)(F)F]

2,6-diamino-N-(4-(2,2,2-trifluoroethoxy)benzyl)-5-(3-(1-(trifluoromethyl)cyclopropyl)-1H-1,2,4-triazol-1-yl)nicotinamide represented by the following formula (VII):

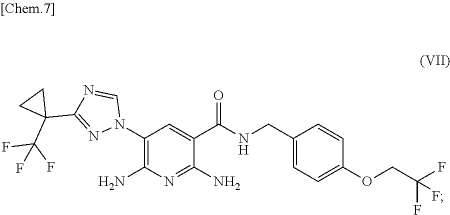 [SMILES: Nc1nc(N)c(-n2cnc(C3(C(F)(F)F)CC3)n2)cc1C(=O)NCc1ccc(OCC(F)(F)F)cc1]

(R)-2,6-diamino-5-(3-(1-fluorocyclopropyl)-1H-1,2,4-triazol-1-yl)-N-(4-((1,1,1-trifluoropropan-2-yl)oxy)benzyl)nicotinamide represented by the following formula (VIII):

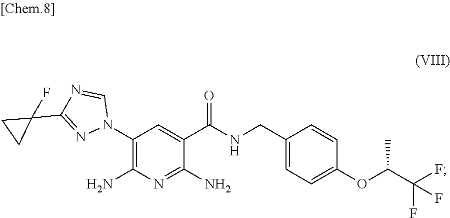 [SMILES: C[C@@H](Oc1ccc(CNC(=O)c2cc(-n3cnc(C4(F)CC4)n3)c(N)nc2N)cc1)C(F)(F)F]

and
2,6-diamino-5-(1-(difluoromethyl)-1H-pyrazol-4-yl)-N-(2-fluoro-4-(2,2,2-trifluoroethoxy)benzyl)nicotinamide represented by the following formula (IX):

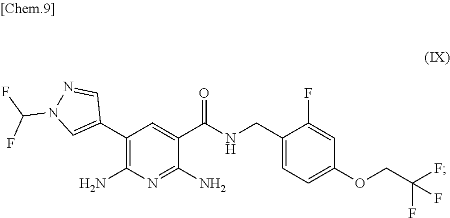 [SMILES: Nc1nc(N)c(-c2cnn(C(F)F)c2)cc1C(=O)NCc1ccc(OCC(F)(F)F)cc1F]

or a pharmaceutically acceptable salt thereof.
<2>
(R)-2,6-diamino-5-(3-(trifluoromethyl)-1H-1,2,4-triazol-1-yl)-N-(4-((1,1,1-trifluoropropan-2-yl)oxy)benzyl)nicotinamide represented by the following formula (I):

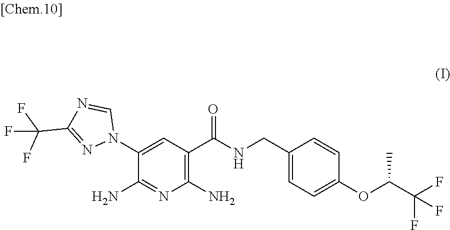 [SMILES: C[C@@H](Oc1ccc(CNC(=O)c2cc(-n3cnc(C(F)(F)F)n3)c(N)nc2N)cc1)C(F)(F)F]

or a pharmaceutically acceptable salt thereof.
<3>
(R)-2,6-diamino-5-(1-(difluoromethyl)-1H-pyrazol-4-yl)-N-(4-((1,1,1-trifluoropropan-2-yl)oxy)benzyl)nicotinamide represented by the following formula (II):

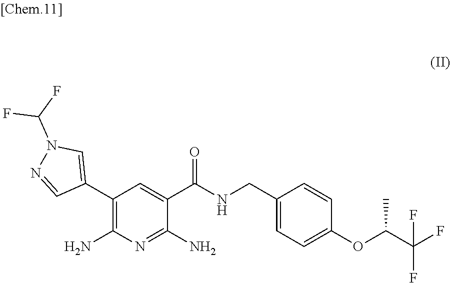 [SMILES: C[C@@H](Oc1ccc(CNC(=O)c2cc(-c3cnn(C(F)F)c3)c(N)nc2N)cc1)C(F)(F)F]

or a pharmaceutically acceptable salt thereof.
<4>
(R)-2,6-diamino-5-(1-(difluoromethyl)-3-methyl-1H-pyrazol-4-yl)-N-(4-((1,1,1-trifluoropropan-2-yl)oxy)benzyl)nicotinamide represented by the following formula (III):

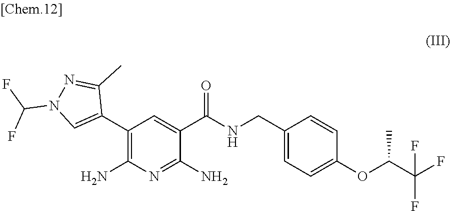 [SMILES: Cc1nn(C(F)F)cc1-c1cc(C(=O)NCc2ccc(O[C@H](C)C(F)(F)F)cc2)c(N)nc1N]

or a pharmaceutically acceptable salt thereof.
<5>
2,6-diamino-N-(4-(2,2,2-trifluoroethoxy)benzyl)-5-(3-(trifluoromethyl)-1H-1,2,4-triazol-1-yl)nicotinamide represented by the following formula (IV):

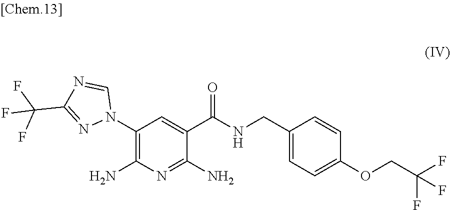 [SMILES: Nc1nc(N)c(-n2cnc(C(F)(F)F)n2)cc1C(=O)NCc1ccc(OCC(F)(F)F)cc1]

or a pharmaceutically acceptable salt thereof.
<6>
(R)-2,6-diamino-5-(3-(difluoromethyl)-1H-1,2,4-triazol-1-yl)-N-(4-((1,1,1-trifluoropropan-2-yl)oxy)benzyl)nicotinamide represented by the following formula (V):

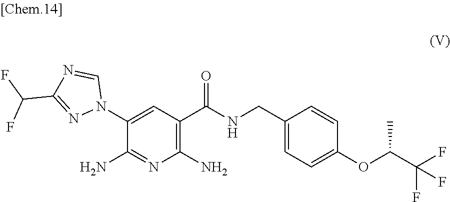 [SMILES: C[C@@H](Oc1ccc(CNC(=O)c2cc(-n3cnc(C(F)F)n3)c(N)nc2N)cc1)C(F)(F)F]

or a pharmaceutically acceptable salt thereof.
<7>
(R)-2,6-diamino-5-(3-(2,2,2-trifluoroethyl)-1H-1,2,4-triazol-1-yl)-N-(4-((1,1,1-trifluoropropan-2-yl)oxy)benzyl)nicotinamide represented by the following formula (VI):

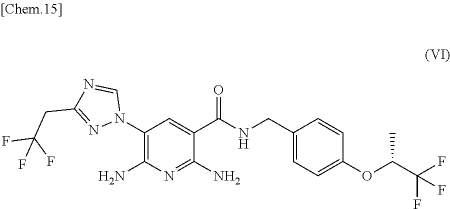 [SMILES: C[C@@H](Oc1ccc(CNC(=O)c2cc(-n3cnc(CC(F)(F)F)n3)c(N)nc2N)cc1)C(F)(F)F]

or a pharmaceutically acceptable salt thereof.
<8>
2,6-diamino-N-(4-(2,2,2-trifluoroethoxy)benzyl)-5-(3-(1-(trifluoromethyl)cyclopropyl)-1H-1,2,4-triazol-1-yl)nicotinamide represented by the following formula (VII):

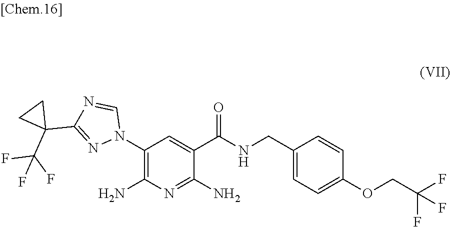 [SMILES: Nc1nc(N)c(-n2cnc(C3(C(F)(F)F)CC3)n2)cc1C(=O)NCc1ccc(OCC(F)(F)F)cc1]

or a pharmaceutically acceptable salt thereof.
<9>
(R)-2,6-diamino-5-(3-(1-fluorocyclopropyl)-1H-1,2,4-triazol-1-yl)-N-(4-((1,1,1-trifluoropropan-2-yl)oxy)benzyl)nicotinamide represented by the following formula (VIII):

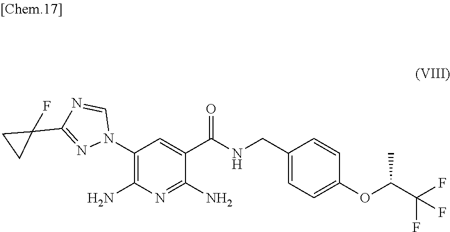 [SMILES: C[C@@H](Oc1ccc(CNC(=O)c2cc(-n3cnc(C4(F)CC4)n3)c(N)nc2N)cc1)C(F)(F)F]

or a pharmaceutically acceptable salt thereof.
<10>
2,6-diamino-5-(1-(difluoromethyl)-1H-pyrazol-4-yl)-N-(2-fluoro-4-(2,2,2-trifluoroethoxy)benzyl)nicotinamide represented by the following formula (IX):

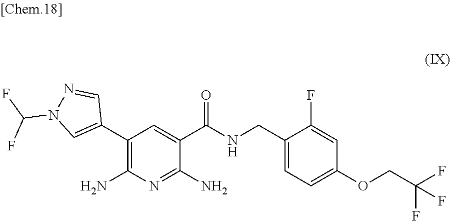 [SMILES: Nc1nc(N)c(-c2cnn(C(F)F)c2)cc1C(=O)NCc1ccc(OCC(F)(F)F)cc1F]

or a pharmaceutically acceptable salt thereof.
<11> a pharmaceutical composition comprising the compound or a pharmaceutically acceptable salt thereof according to any one of <1> to <10>.
<12> an antimalarial agent comprising the compound or the pharmaceutically acceptable salt thereof according to any one of <1> to <10>.
<13> a malaria prophylactic agent comprising the compound or the pharmaceutically acceptable salt thereof according to any one of <1> to <10>.
<14> a method of treating or preventing malaria, comprising administering to a subject the compound or a pharmaceutically acceptable salt thereof according to any one of <1> to <10>.
<15> the compound or a pharmaceutically acceptable salt thereof according to any one of <1> to <10> for use as a medicament.
<16> the compound or a pharmaceutically acceptable salt thereof according to any one of <1> to <10> for use in the treatment or prevention of malaria.
<17> use of the compound or a pharmaceutically acceptable salt thereof according to any one of <1> to <10> for the manufacture of a pharmaceutical composition for the treatment or prevention of malaria.

### Advantageous Effects of Invention

The diamino pyridine compounds represented by formulas (I) to (IX) (hereinafter referred to as compounds (I) to (IX) or the compound(s) of the present invention in general) or pharmaceutically acceptable salts thereof according to the present invention have antimalarial activation effect, as shown in activity data in Pharmacological Test Examples below. Since compounds (I) to (IX) of the invention lead to an antimalarial action, and thus have a potential use as a prophylactic agent and/or therapeutic agent for tropical malaria, tertian malaria, quartan malaria and/or ovale malaria.

### DESCRIPTION OF EMBODIMENTS

The present invention will be hereinafter described in detail.

In the present specification, the structural formulas of the compounds may represent specific isomers for convenience; however, the present invention may include rotational isomers and tautomers, as well as isomeric mixtures, is not limited to the formulas described for convenience, and may be any of the isomers or a mixture containing the isomers in any proportion.

Further, polymorphic crystals may also exist; however, the present invention is also not limited to any of them and may be a singly crystal form or a mixture thereof. Moreover, the present invention also includes amorphous forms, and the compounds according to the present invention include anhydrates and solvates (particularly hydrates).

The present invention also includes isotope-labeled compounds of the compounds (1) to (IX). The isotope-labeled compounds are the same as the compounds (I) to (IX), except that one or more atoms are replaced by one or more atoms having an atomic mass or mass number different from those generally found in nature. Examples of isotopes that can be incorporated into the compounds of the present invention include isotopes of hydrogen, carbon, nitrogen, oxygen, fluorine, phosphorus, sulfur, iodine, and chlorine, and specifically include 2H, 3H, 11C, 14C, 15N, 18O, 18F, 32P, 35S, 123I, 125I, and the like.

The above isotope-labeled compounds, for example, compounds into which radioactive isotopes, such as 3H and/or 14C, are incorporated, are useful for the tissue distribution assay of medicines and/or substrates. 3H and 14C are considered to be useful because of the ease of the preparation and detection thereof. Isotopes 11C and 18F are considered to be useful for PET (positron emission tomography), isotope 125I is considered to be useful for SPECT (single-photon emission computed tomography), and all of them are useful for brain imaging. Replacement by heavier isotopes, such as 2H, results in some types of therapeutic advantages, including an increase in the in vivo half-life period or a decrease in the required dose due to higher metabolic stability, and is therefore considered to be useful under certain situations. The above isotope-labeled compounds can be similarly prepared by carrying out the procedures disclosed in the following Examples using easily usable reagents labeled with isotopes in place of reagents not labeled with isotopes.

The “pharmaceutically acceptable salts” in the present specification are not particularly limited as long as they are salts formed with the compounds according to the present invention, and specific examples include acid addition salts, such as inorganic acid salts, organic acid salts, and acidic amino acid salts.

The “pharmaceutically acceptable salt” in the present specification is any salt formed in a suitable ratio unless there is any especially limiting description, and the number of acid molecules per molecule of the compound in the formed salt is not particularly limited; however, it is preferable that the number of acid molecules per molecule of the compound be about 0.5 to about 2, and it is more preferable that the number of acid molecules per molecule of the compound be about 0.5, about 1, or about 2.

Preferable examples of the inorganic acid salts include hydrochloride, hydrobromide, sulfate, nitrate, and phosphate; and preferable examples of organic acid salts include acetate, succinate, fumarate, maleate, tartrate, citrate, lactate, stearate, benzoate, methanesulfonate, p-toluenesulfonate, and benzenesulfonate.

Preferable examples of the acidic amino acid salts include aspartate and glutamate.

When the compounds (I) to (IX) according to the present invention are obtained in a free form, they can be converted into salts that may be formed by the compounds (I) to (IX) or hydrates thereof in accordance with a conventional method.

When the compounds (I) to (IX) according to the present invention are obtained as salts of the compounds (1) to (IX) or hydrates of the compounds (I) to (IX), they can be converted into free forms of the compounds (I) to (IX) in accordance with a conventional method.

Moreover, various isomers (e.g., optical isomers, rotational isomers, stereoisomers, etc.) obtained from the compounds (I) to (IX) according to the present invention can be purified and isolated by general separation means, such as recrystallization, diastereomeric salt method, enzymatic resolution method, and various chromatographic techniques (e.g., thin-layer chromatography, column chromatography, gas chromatography, etc.).

Pharmaceutical Preparation

The pharmaceutical composition according to the present invention can be produced by mixing pharmaceutically acceptable additives with a compound selected from the group of compounds (I) to (IX) or pharmaceutically acceptable salts thereof. The pharmaceutical composition according to the present invention can be produced by a known method, for example, the method described in the General Rules for Preparations of The Japanese Pharmacopoeia Seventeenth Edition.

The pharmaceutical composition according to the present invention can be appropriately administered to a patient depending on the dosage form thereof.

The dose of the compounds (I) to (IX) according to the present invention or pharmaceutically acceptable salts thereof varies depending on the severity of symptoms, age, sex, body weight, dosage form, type of salt, specific type of disease, and other conditions; however, in general, the dose for an adult per day by oral administration is about 30 micro g to 10 g, preferably 100 micro g to 5 g, and more preferably 100 micro g to 1 g; the dose for an adult per day by injection administration is about 30 micro g to 1 g, preferably 100 micro g to 500 mg, and more preferably 100 micro g to 300 mg; and the above dose is administered once or several times.

The term “malaria” includes disease and conditions related to an infection by Plasmodium.

As used herein, “treatment” and “treating” and the like generally mean obtaining a desired pharmacological and physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein covers any treatment of a disease in a mammal, particularly a human, and includes: (a) preventing the disease from occurring in a subject which may be predisposed to the disease but has not yet been diagnosed as having it; (b) inhibiting the disease, i.e., arresting its development; or relieving the disease, i.e., causing regression of the disease and/or its symptoms or conditions.

The term “effective amount” includes “prophylaxis-effective amount” as well as “treatment-effective amount”.

The term “prophylaxis-effective amount” refers to a concentration of compound of the present invention that is effective in inhibiting, decreasing the likelihood of the disease by malarial parasites, or preventing malarial infection or preventing the delayed onset of the disease by malarial parasites, when administered before infection, i.e. before, during and/or slightly after the exposure period to malarial parasites.

The term “prophylaxis” includes causal prophylaxis, i.e. antimalarial activity comprising preventing the pre-erythrocytic development of the parasite, suppressive prophylaxis, i.e. antimalarial activity comprising suppressing the development of the blood stage infection and terminal prophylaxis, i.e. antimalarial activity comprising suppressing the development of intra-hepatic stage infection. This term includes primary prophylaxis (i.e. preventing initial infection) where the antimalarial compound is administered before, during and/or after the exposure period to malarial parasites and terminal prophylaxis (i.e. to prevent relapses or delayed onset of clinical symptoms of malaria) when the antimalarial compound is administered towards the end of and/or slightly after the exposure period to malarial parasites but before the clinical symptoms. Typically, against P. falciparum infections, suppressive prophylaxis is used whereas against P. vivax or a combination of P. falciparum and P. vivax, terminal prophylaxis is used.

Likewise, the term “treatment-effective amount” refers to a concentration of compound that is effective in treating malaria infection, e.g. leads to a reduction in parasite numbers in blood following microscopic examination when administered after infection has occurred.

The term “subject” as used herein refers to mammals. For examples, mammals contemplated by the present invention include humans and the like.

Compositions

The invention provides pharmaceutical compositions useful for the prophylaxis or treatment of malaria. The invention further provides methods for treating a mammalian patient, and most preferably a human patient, who is suffering from malaria.

In another particular embodiment, is provided a pharmaceutical composition comprising at least one compound of the present invention and a pharmaceutically acceptable carrier, diluent or excipient.

In another particular embodiment, is provided a pharmaceutical composition comprising at least one compound of the present invention and a further antimalarial agent as defined below.

In another particular embodiment, is provided a pharmaceutical composition comprising at least one compound of the present invention and at least one further antimalarial agent selected from the group consisting of artemisinin and its derivatives such as artemisinin and its derivatives (such as artemether, artesunate or dihydroartemisinin), chloroquine, hydroxychloroquine, quinine, quinidine, mefloquine, amodiaquine, atovaquone/proguanil, clindamycin, doxycycline, lumefantrine, piperaquine, pyronaridine, halofantrine, pyrimethamine-sulfadoxine, primaquine, quinacrine, ferroquine, tafenoquine, arterolane, Spiro[3H-indole-3,1′-[1H]pyrido[3,4-b]indol]-2(1H)-one, 5,7′-dichloro-6′-fluoro-2′,3′,4′,9′-tetrahydro-3′-methyl-,(1′R,3′S)-] (Cipargamin, KAE609, CAS Registry Number: 1193314-23-6), 2-(1,1-difluoroethyl)-5-methyl-N-[4-(pentafluoro-lambda6-sulfanyl)phenyl]-[1,2,4]triazolo[1,5-a] pyrimidin-7-amine (DSM265, CAS Registry Number: 1282041-94-4), Morpholine, 4-[2-(4-cis-dispiro[cyclohexane-1,3′-[1,2,4]trioxolane-5′,2″-tricyclo[3.3.1.13,7]decan]-4-ylphenoxy)ethyl]-] (Artefenomel, OZ439, CAS Registry Number: 1029939-86-3), 4-Quinolinecarboxamide, 6-fluoro-2-[4-(4-morpholinylmethyl)phenyl]-N-[2-(1-pyrrolidinyl)ethyl]-(DDD107498, CAS Registry Number: 1469439-69-7), Ethanone, 2-amino-1-[2-(4-fluorophenyl)-3-[(4-fluorophenyl)amino]-5,6-dihydro-8,8-dimethylimidazo[1,2-a]pyrazin-7(8H)-yl]- (Ganaplacide, KAF-156, CAS Registry Number 1261113-96-5), 5-[4-(Methylsulfonyl)phenyl]-6′-(trifluoromethyl)[3,3′-bipyridin]-2-amine (MMV390048, CAS Registry Number: 1314883-11-8), 4(1H)-Quinolinone, 6-chloro-7-methoxy-2-methyl-3-[4-[4-(trifluoromethoxy)phenoxy]phenyl]- (ELQ-300, CAS Registry Number: 1354745-52-0) and 4-Isoquinolinecarboxamide, N-(3-cyano-4-fluorophenyl)-1,2,3,4-tetrahydro-1-oxo-3-(3-pyridinyl)-2-(2,2,2-trifluoroethyl)-, (3S,4S)- (SJ-733, CAS Registry Number 1424799-20-1).

The Pharmaceutical compositions of the present invention can comprise at least one compound of the present invention in any form described herein. The pharmaceutical compositions of the present invention may further comprise one or more pharmaceutically acceptable additional ingredient(s), such as alum, stabilizers, antimicrobial agents, buffers, coloring agents, flavoring agents, adjuvants, and the like.

The compound of the present invention, together with a conventionally employed adjuvant, carrier, diluent or excipient may be placed into the form of pharmaceutical compositions and unit dosages thereof, and in such form may be employed as solids, such as tablets or filled capsules, or liquids such as solutions, suspensions, emulsions, elixirs, or capsules filled with the same, all for oral use, or in the form of sterile injectable solutions for parenteral (including subcutaneous) use. Such pharmaceutical compositions and unit dosage forms thereof may comprise ingredients in conventional proportions, with or without additional active compounds or principles, and such unit dosage forms may contain any suitable effective amount of the active ingredient commensurate with the intended dosage range to be employed. The pharmaceutical compositions of the present invention are preferably oral.

The pharmaceutical compositions of the present invention may be liquid formulations, including, but not limited to, aqueous or oily suspensions, solutions, emulsions, syrups, and elixirs. Liquid forms suitable for oral administration may include a suitable aqueous or non-aqueous vehicle with buffers, suspending agents, dispersing agents, colorants, flavors and the like. The pharmaceutical compositions may also be formulated as a dry product for reconstitution with water or other suitable vehicle before use. Such liquid preparations may contain additives, including, but not limited to, suspending agents, emulsifying agents, non-aqueous vehicles and preservatives. Suspending agents include, but are not limited to, sorbitol syrup, methyl cellulose, glucose/sugar syrup, gelatin, hydroxyethylcellulose, carboxymethyl cellulose, aluminum stearate gel, and hydrogenated edible fats. Emulsifying agents include, but are not limited to, lecithin, sorbitan monooleate, and acacia. Non-aqueous vehicles include, but are not limited to, edible oils, almond oil, fractionated coconut oil, oily esters, propylene glycol, and ethyl alcohol. Preservatives include, but are not limited to, methyl or propyl p-hydroxybenzoate and sorbic acid. Further materials as well as processing techniques and the like are set out in out in Part 5 of Remington's “The Science and Practice of Pharmacy”, 22nd Edition, 2012, University of the Sciences in Philadelphia, Lippincott Williams & Wilkins, which is incorporated herein by reference. Solid pharmaceutical compositions of the present invention may be in the form of tablets or lozenges formulated in a conventional manner. For example, tablets and capsules for oral administration may contain conventional excipients including, but not limited to, binding agents, fillers, lubricants, disintegrants and wetting agents. Binding agents include, but are not limited to, syrup, acacia, gelatin, sorbitol, tragacanth, mucilage of starch and polyvinylpyrrolidone. Fillers include, but are not limited to, lactose, sugar, microcrystalline cellulose, maize starch, calcium phosphate, and sorbitol. Lubricants include, but are not limited to, magnesium stearate, stearic acid, talc, polyethylene glycol, and silica. Disintegrants include, but are not limited to, potato starch and sodium starch glycollate. Wetting agents include, but are not limited to, sodium lauryl sulfate. Tablets may be coated according to methods well known in the art.

Injectable pharmaceutical compositions are typically based upon injectable sterile saline or phosphate-buffered saline or other injectable carriers known in the art.

Pharmaceutical compositions of the present invention may also be formulated as suppositories, which may contain suppository bases including, but not limited to, cocoa butter or glycerides. Pharmaceutical compositions of the present invention may also be formulated for inhalation, which may be in a form including, but not limited to, a solution, suspension, or emulsion that may be administered as a dry powder or in the form of an aerosol using a propellant, such as dichlorodifluoromethane or trichlorofluoromethane. Pharmaceutical compositions of the present invention may also be formulated transdermal formulations comprising aqueous or non-aqueous vehicles including, but not limited to, creams, ointments, lotions, pastes, medicated plaster, patch, or membrane.

Pharmaceutical compositions of the present invention may also be formulated for parenteral administration, including, but not limited to, by injection or continuous infusion. Formulations for injection may be in the form of suspensions, solutions, or emulsions in oily or aqueous vehicles, and may contain formulation agents including, but not limited to, suspending, stabilizing, and dispersing agents. Pharmaceutical compositions may also be provided in a powder form for reconstitution with a suitable vehicle including, but not limited to, sterile, pyrogen-free water.

Pharmaceutical compositions of the present invention may also be formulated as a depot preparation, which may be administered by implantation or by intramuscular injection. Pharmaceutical compositions may be formulated with suitable polymeric or hydrophobic materials (as an emulsion in an acceptable oil, for example), ion exchange resins, or as sparingly soluble derivatives (as a sparingly soluble salt, for example).

Pharmaceutical compositions of the present invention may also be formulated as a liposome preparation. The liposome preparation can comprise liposomes which penetrate the cells of interest or the stratum corneum, and fuse with the cell membrane, resulting in delivery of the contents of the liposome into the cell. Other suitable formulations can employ niosomes. Niosomes are lipid vesicles similar to liposomes, with membranes consisting largely of non-ionic lipids, some forms of which are effective for transporting compounds across the stratum corneum.

The compounds of the present invention can also be administered in sustained release forms or from sustained release drug delivery systems. A description of representative sustained release materials can also be found in the incorporated materials in Remington's Pharmaceutical Sciences.

Mode of Administration

Pharmaceutical compositions of the present invention may be administered in any manner, including, but not limited to, orally, parenterally, sublingually, transdermally, vaginally, rectally, transmucosally, topically, via inhalation, via buccal or intranasal administration, or combinations thereof. Parenteral administration includes, but is not limited to, intravenous, intra-arterial, intra-peritoneal, subcutaneous, intramuscular, intra-thecal, and intra-articular. Pharmaceutical compositions of the present invention may also be administered in the form of an implant, which allows slow release of the compositions as well as a slow controlled i.v. infusion. In a preferred embodiment, Pharmaceutical compositions of the present invention are administered orally.

Combination

The compound or pharmaceutical composition of the present invention can be administered alone or in combination with a co-agent useful in the treatment of malaria, such as substances useful in the treatment and/or prevention of malaria e.g. for example a co-agent including, but not limited to, artemisinin and its derivatives such as artemisinin and its derivatives (such as artemether, artesunate or dihydroartemisinin), chloroquine, hydroxychloroquine, quinine, quinidine, mefloquine, amodiaquine, atovaquone/proguanil, clindamycin, doxycycline, lumefantrine, piperaquine, pyronaridine, halofantrine, pyrimethamine-sulfadoxine, primaquine, quinacrine, ferroquine, tafenoquine, arterolane, Spiro[3H-indole-3,1′-[1H]pyrido[3,4-b]indol]-2(1H)-one, 5,7′-dichloro-6′-fluoro-2′,3′,4′,9′-tetrahydro-3′-methyl-,(1′R,3'S)-] (Cipargamin, KAE609, CAS Registry Number: 1193314-23-6), 2-(1,1-difluoroethyl)-5-methyl-N-[4-(pentafluoro-lambda6-sulfanyl)phenyl]-[1,2,4]triazolo[1,5-a] pyrimidin-7-amine (DSM265, CAS Registry Number: 1282041-94-4). Morpholine, 4-[2-(4-cis-dispiro[cyclohexane-1,3′-[1,2,4]trioxolane-5′,2″-tricyclo[3.3.1.13.7]decan]-4-ylphenoxy)ethyl]-J (Artefenomel, OZ439, CAS Registry Number: 1029939-86-3), 4-Quinolinecarboxamide, 6-fluoro-2-[4-(4-morpholinylmethyl)phenyl]-N-[2-(1-pyrrolidinyl)ethyl]-(DDD107498, CAS Registry Number: 1469439-69-7), Ethanone, 2-amino-1-[2-(4-fluorophenyl)-3-[(4-fluorophenyl)amino]-5,6-dihydro-8,8-dimethylimidazo[1,2-a]pyrazin-7(8H)-yl]- (Ganaplacide, KAF-156, CAS Registry Number 1261113-96-5), 5-[4-(Methylsulfonyl)phenyl]-6′-(trifluoromethyl)[3,3′-bipyridin]-2-amine (MMV390048, CAS Registry Number: 1314883-11-8), 4(1H)-Quinolinone, 6-chloro-7-methoxy-2-methyl-3-[4-[4-(trifluoromethoxy)phenoxy]phenyl]- (ELQ-300, CAS Registry Number: 1354745-52-0) and 4-Isoquinolinecarboxamide, N-(3-cyano-4-fluorophenyl)-1,2,3,4-tetrahydro-1-oxo-3-(3-pyridinyl)-2-(2,2,2-trifluoroethyl)-, (3S,4S)- (SJ-733, CAS Registry Number 1424799-20-1).

The invention encompasses the administration of a compound or a pharmaceutical composition of the present invention, wherein the compound or the pharmaceutical composition is administered to an individual prior to, simultaneously or sequentially with another therapeutic regimen or a co-agent useful in the treatment of malaria (e.g. multiple drug regimens), in an effective amount. The compound or the pharmaceutical composition that are administered simultaneously with said co-agent can be administered in the same or different composition(s) and by the same or different route(s) of administration.

The invention provides a use of a compound or a method according to the invention wherein the compound is to be administered in combination with a co-agent useful in the treatment of malaria. In another embodiment, the invention provides a pharmaceutical composition comprising a compound according to the invention in combination with a co-agent useful in the treatment of malaria.

The compounds of the present invention can be used as chemical probes for capturing the target proteins of bioactive low-molecular-weight compounds. That is, the compounds of the present invention can be converted into affinity chromatography probes, photoaffinity probes, etc., by introducing labeling groups, linkers, or the like into a moiety different from a structural moiety essential for the development of the activity of the compounds using a method described, for example, in J. Mass Spectrum. Soc. Jpn. Vol. 51, No. 5, 2003, pp. 492-498, WO2007/139149, or the like.

Examples of labeling groups, linkers, etc., used in chemical probes include groups shown in the group consisting of the following (1) to (5):

(1) protein-labeling groups, such as photoaffinity-labeling groups (e.g., a benzoyl group, a benzophenone group, an azide group, a carbonylazide group, a diaziridine group, an enone group, a diazo group, a nitro group, etc.) and chemical affinity groups (e.g., a ketone group in which the alpha carbon atom is replaced by a halogen atom, a carbamoyl group, an ester group, an alkylthio group, a Michael acceptor such as alpha,beta-unsaturated ketone or ester, and an oxirane group);
(2) cleavable linkers, such as —S—S—, —O—Si—O—, monosaccharides (a glucose group, a galactose group, etc.), or disaccharides (lactose, etc.); and oligopeptide linkers cleavable by enzyme reaction;
(3) fishing tag groups, such as biotin and a 3-(4,4-difluoro-5,7-dimethyl-4H-3a,4a-diaza-4-bora-s-indacen-3-yl)propionyl group;
(4) radioactive labeling groups, such as 125I, 32P, 3H, and 14C; fluorescent labeling groups, such as fluorescein, rhodamine, dansyl, umbelliferone, 7-nitrofurazanyl, and a 3-(4,4-difluoro-5,7-dimethyl-4H-3a,4a-diaza-4-bora-s-indacen-3-yl)propionyl group; chemiluminescent groups, such as luciferin and luminol; and markers capable of detecting heavy metal ions, such as lanthanoid metal ions and radium ions; or
(5) groups to be attached to solid carriers, such as glass beads, glass beds, microtiter plates, agarose beads, agarose beds, polystyrene beads, polystyrene beds, nylon beads, and nylon beds.

Probes prepared by introducing labeling groups, etc., selected from the group consisting of the above (1) to (5) into the compounds of the present invention by the methods described in the above documents or the like can be used as chemical probes for identifying labeled proteins useful to search novel drug design targets, etc.

### EXAMPLES

The chemical names for the compounds in the following examples were created based on the chemical structures using “E-Notebook 2014” version 13 (PerkinElmer Co., Ltd.). Silica gel chromatography was carried out using pre-packed silica gel cartridges on a Biotage(Trademark) Isolera Four(Trademark).

¹H NMR spectra chemical shifts are expressed in ppm relative to an internal standard, tetramethylsilane (ppm=0.00). The following abbreviations have been used: br=broad signal, s=singlet, d=doublet, dd=double doublet, t=triplet, q=quartet, app spt=apparent septet, m=multiplet, or any combination thereof. ¹H-NMR was measured using a Bruker AVIII (600 MHz).

The ee determinations were carried out on a Shimadzu Chiral HPLC instrument.

HPLC purifications were carried out on a Waters MDAP system with a Waters SQD mass

Moreover, the abbreviations used in the present specification are well-known and common to a person skilled in the art. In the present specification, the following abbreviations are used.

app: apparent
BH3.THF: Borane-tetrahydrofuran complex
c.: concentrated

### DCM: Dichloromethane

### DIPEA: N,N-Diisopropylethylamine

DMF-DMA: N,N-Dimethylformamide dimethyl acetal

### Dppf: 1,1′-Ferrocenediyl-bis(diphenylphosphine)

EDC.HCl: N-(3-Dimethylaminopropyl)-N′-ethylcarbodiimide hydrochloride

### HOBT: 1-Hydroxybenzotriazole

HPLC: High pressure liquid chromatography

### IMS: Industrial Methylated Spirit

LCMS: Liquid chromatography-Mass Spectroscopy

### MDAP: Mass Directed Autopurification

### 2-MeTHF: 2-Methyl Tetrahydrofuran

TBME: tert-Butyl methyl ether

### NBS: N-Bromosuccinimide

### NCS: N-Chlorosuccinimide

### NIS: N-Iodosuccinimide

### Pd(PPh3)4: Tetrakis(triphenylphosphine)palladium(0)

### PhMe: Toluene

RT: Room temperature
SPhos: 2-Dicyclohexylphosphino-2′,6′-dimethoxybiphenyl
SQD: Single quadrupole detection
TBTU: O-(Benzotriazol-1-yl)-N,N,N′,N′-tetramethyluronium tetrafluoroborate

### THF: Tetrahydrofuran

The compounds (I) to (IX) of the present invention can be produced by, for example, the methods described in the following Examples, and the effects of the compounds can be confirmed by the methods described in the following Test Examples. However, these are just examples, and the present invention is not limited to the following specific examples in any case and may be modified within a range that does not depart from the scope of the present invention.

### Production Example 1

### Preparation of (R)-2,6-diamino-5-(3-(trifluoromethyl)-1H-1,2,4-triazol-1-yl)-N-(4-((1,1,1-trifluoropropan-2-yl)oxy)benzyl)nicotinamide (I)

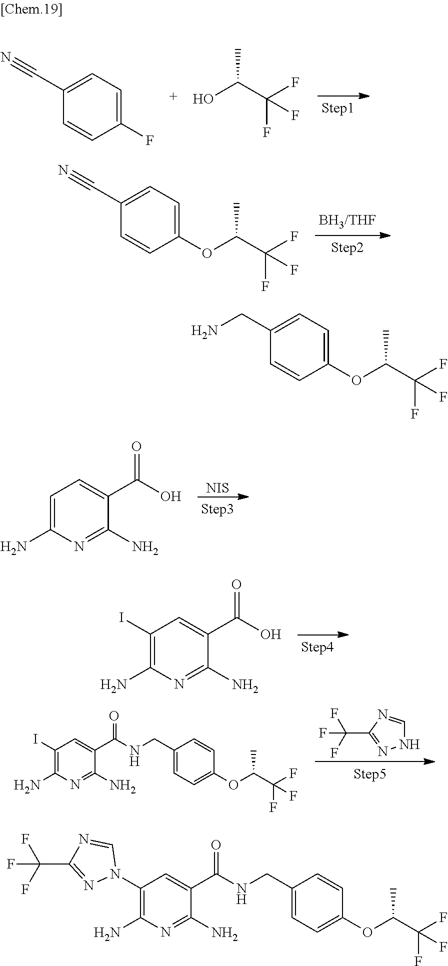

### Step 1: Preparation of (R)-4-((1,1,1-trifluoropropan-2-yl)oxy)benzonitrile

(R)-1,1,1-trifluoropropan-2-ol (2.00 ml, 21.7 mmol) was added to a stirring suspension of potassium tert-butoxide (3.47 g, 30.9 mmol) in THF (35 mL), under nitrogen, at 0° C. A solution of 4-fluorobenzonitrile (2.5 g, 20.6 mmol) in THF (35 mL) was added dropwise, after 10 min. The reaction was allowed to warm to RT after 1 h. The reaction was partitioned between EtOAc/water, the organic portion washed with brine, dried over Na2SO4 and evaporated to give a crude oil (5.4 g). Column chromatography (taken up in c-Hex, 0-50% EtOAc/c-Hex, 50 g Silica-gel) gave the title compound (4.27 g).

1H NMR (600 MHz, CDCl3) delta ppm 1.54 (d, J=6.4 Hz, 3H) 4.74 (t, J=6.1 Hz, 1H) 7.02 (d, J=8.8 Hz, 2H) 7.63 (d, J=8.8 Hz, 2H)

### Step 2: Preparation of (R)-(4-((1,1,1-trifluoropropan-2-yl)oxy)phenyl)methanamine

To a stirred solution of (R)-4-((1,1,1-trifluoropropan-2-yl)oxy)benzonitrile (4.1 g, 19.1 mmol) in THF (39 ml) at 0° C. was added a 1 M borane tetrahydrofuran complex (38.1 ml, 38.1 mmol) in THF dropwise. The reaction was stirred for 1 h at 0° C., allowed to warm to RT then heated to 65° C. The reaction was allowed to cool to RT, 2 M HCl (39.0 ml, 78.0 mmol) added dropwise and the mixture stirred at 65° C. for 2 h then at 100° C. for a further 2 h. The reaction mixture was allowed to cool, diluted with MeOH, loaded onto a SCX-2 cartridge, washed with MeOH and the product eluted with 2 M NH3/MeOH. Evaporation of the combined NH3/MeOH washings gave the title compound (3.76 g).

1H NMR (600 MHz, CDCl3) delta ppm 1.49 (d, J=6.6 Hz, 3H) 3.83 (s, 2H) 4.61 (t, J=6.3 Hz, 1H) 6.93 (d, J=8.6 Hz, 2H) 7.24-7.27 (m, 2H)

### Step 3: Preparation of 2,6-diamino-5-iodonicotinic acid

NIS (1.63 g, 7.24 mmol) was added portionwise over 5 min to a stirring suspension of 2,6-diaminonicotinic acid (1.01 g, 6.60 mmol) in DMF (20 ml) at RT. The starting material was heated and sonicated to break into smaller bits. The reaction was diluted with 1 M NaOH and partitioned with EtOAc. The layers were separated and the aqueous portion acidified to pH 6 with c. HCl. A precipitate formed, which was filtered, washed with water, EtOAc and dried under vacuum to give the title compound (1.28 g).

1H NMR (600 MHz, DMSO-d6) delta ppm 6.35 (br s, 2H) 6.97 (br s, 2H) 8.01 (s, 1H) 12.15 (br s, 1H)

### Step 4: Preparation of (R)-2,6-diamino-5-iodo-N-(4-((1,1,1-trifluoropropan-2-yl)oxy)benzyl)nicotinamide

To a stirred solution of 2,6-diamino-5-iodonicotinic acid (1 g, 3.58 mmol) in DMSO (10 mL) was added HOBT H2O (0.659 g, 4.30 mmol) and EDC HCl (0.824 g, 4.30 mmol). After 5 min, a solution of (R)-(4-((1,1,1-trifluoropropan-2-yl)oxy)phenyl)methanamine (0.825 g, 3.76 mmol) and triethylamine (2.00 ml, 14.3 mmol) in DMSO (10 mL) was added dropwise and the resulting solution stirred at RT, 20 h. The reaction was partitioned between EtOAc/water, the organic portion washed with brine, dried over Na2SO4 and evaporated to give an orange residue (2.15 g). Purification by column chromatography (20-80% EtOAc/c-Hex, 25 g KP-Sil) gave the title compound (1.08 g).

1H NMR (600 MHz, DMSO-d6) delta ppm 1.39 (d, J=6.4 Hz, 3H) 4.30 (d, J=5.7 Hz, 2H) 5.16 (app spt, J=6.5 Hz, 1H) 6.13 (br s, 2H) 7.02 (d, J=8.6 Hz, 2H) 7.07 (br s, 2H) 7.23 (d, J=8.4 Hz, 2H) 8.11 (s, 1H) 8.51 (t, J=5.8 Hz, 1H)

### Step 5: Preparation of

### (R)-2,6-diamino-5-(3-(trifluoromethyl)-1H-1,2,4-triazol-1-yl)-N-(4-((1,1,1-trifluoropropan-2-yl)oxy)benzyl)nicotinamide (I)

(R)-2,6-diamino-5-iodo-N-(4-((1,1,1-trifluoropropan-2-yl)oxy)benzyl)nicotinamide (120 mg, 0.25 mmol), 3-(trifluoromethyl)-1H-1,2,4-triazole (47.9 mg, 0.35 mmol), Copper(I) iodide (52.3 mg, 0.275 mmol), N,N′-Dimethylcyclohexane-1,2-diamine (0.043 ml, 0.275 mmol) and Potassium phosphate (106 mg, 0.50 mmol) in DMF (2 ml) were degassed with nitrogen and heated at 120° C., 18 h (sealed tube). The reaction mixture was diluted with MeOH, loaded onto SCX-2. Washed with MeOH, eluted with 2 M NH3/MeOH/DCM to give a brown oil (0.14 g). Purification by column chromatography (20-100% EtOAc/c-Hex, KP-Sil). Combine, evaporate relevant fractions to give the title compound (21 mg).

1H NMR (600 MHz, DMSO-d6) delta ppm 1.38 (d, J=6.4 Hz, 3H) 4.31 (d, J=5.7 Hz, 2H) 5.15 (dt, J=12.8, 6.4 Hz, 1H) 6.39 (s, 2H) 7.01 (d, J=8.4 Hz, 2H) 7.23 (d, J=8.4 Hz, 2H) 7.42 (br s, 2H) 7.92 (s, 1H) 8.41 (br t, J=5.8 Hz, 1H) 8.97 (s, 1H)

### Preparation of (R)-2,6-diamino-5-(3-(trifluoromethyl)-1H-1,2,4-triazol-1-yl)-N-(4-((1,1,1-trifluoropropan-2-yl)oxy)benzyl)nicotinamide (I)

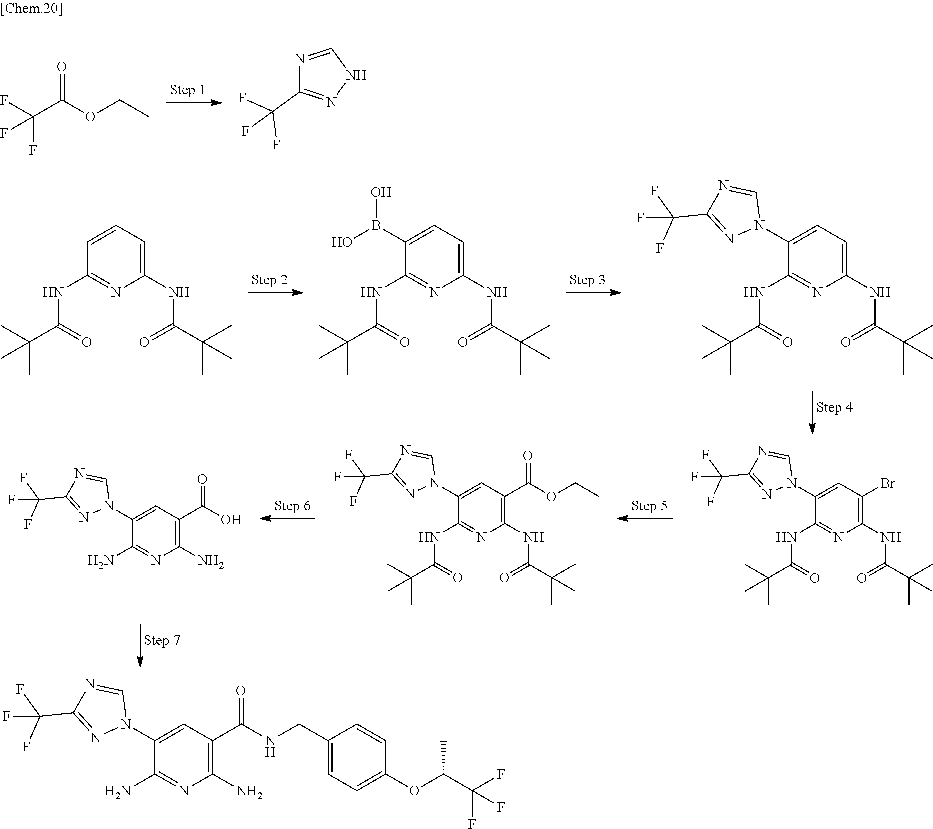

### Step 1: Preparation of 3-(trifluoromethyl)-1H-1,2,4-triazole

To a stirred solution of hydrazine hydrate (100 mL, 2052 mmol) in EtOH (2.6 L) at 10° C. was added ethyl 2,2,2-trifluoroacetate (257 mL, 2161 mmol) (CAS 1194-02-1. Fluorochem) dropwise. The mixture was stirred for 16 h at RT. Iminoformamide acetate (247 g, 2377 mmol) was added and the mixture heated to reflux for 9 h. AcOH (148 mL, 2585 mmol) was added and the EtOH removed by evaporation. The resulting solution was basified with a sat. aqueous NaHCO₃ and extracted with EtOAc (3×500 mL). The combined organic extracts were dried (Na₂SO₄), filtered and evaporated. The residue was triturated with n-heptane, collected by filtration, washed with n-heptane (100 mL) and dried under vacuum to afford the title compound (216 g). LCMS: m/z 138 [M+H]⁺. ¹H NMR (600 MHz, DMSO-d₆) delta ppm 8.84 (s, 1H) 13.99 (br s, 1H).

### Step 2: Preparation of (2,6-dipivalamidopyridin-3-yl)boronic acid

To a stirred solution of N,N′-(pyridine-2,6-diyl)bis(2,2-dimethylpropanamide) (80 g, 288 mmol) in THF (1 L) under N₂ at −70° C. was added n-BuLi (2.5 M in hexane, 400 mL, 1000 mmol) dropwise. The mixture was warmed to 0° C. and stirred for 16 h, then cooled to −60° C. and triisopropyl borate (233 mL, 1009 mmol) was added dropwise. The mixture was allowed to warm to RT and stirred for 90 mins then cooled to 0° C. and sat. aqueous NH₄Cl (600 mL) was added dropwise. The resulting mixture was partitioned between H₂O (600 mL) and 2-MeTHF (200 mL) and the layers separated. The aqueous layer was extracted with 2-MeTHF (2×1 L), the combined organic layers were washed with sat. aqueous NH₄Cl (2×500 mL), dried over Na₂SO₄, filtered and evaporated. The residue was stirred in PhMe (1 L) at RT for 16 h, forming a precipitate which was collected by filtration, washed with cold PhMe (200 mL) and dried under vacuum to afford the title compound (83.2 g). LCMS: m/z 322 [M+H]⁺. ¹H NMR (600 MHz, DMSO-d₆) delta ppm 1.17 (s, 9H) 1.23 (s, 9H) 7.69 (d, J=7.89 Hz, 1H) 7.92 (br d, J=7.89 Hz, 1H) 9.17 (s, 1H) 11.43 (br s, 1H).

### Step 3: Preparation of N,N′-(3-(3-(trifluoromethyl)-1H-1,2,4-triazol-1-yl)pyridine-2,6-diyl)bis(2,2-dimethylpropanamide)

To a stirred solution of (2,6-dipivalamidopyridin-3-yl)boronic acid (30 g, 93.4 mmol) and DMF (600 mL) was added (trifluoromethyl)-1H-1,2,4-triazole (19.2 g, 140 mmol), copper(II) acetate (1.70 g, 9.34 mmol) and pyridine (18.9 mL, 234 mmol). A low vacuum was placed on the three-neck flask allowing air to pass through a drying tube containing anhydrous CaCl₂ and into the solution through a sparging tube. The mixture was heated to 60° C. for 22 h, then cooled and poured into ice water (2 L). After 30 mins a precipitate was collected by filtration, washed with H₂O (200 mL) and n-heptane (200 mL) and dried under vacuum to afford the title compound (32.0 g). LCMS: m/z 413 [M+H]⁺. ¹H NMR (600 MHz, DMSO-d₆) delta ppm 1.03 (s, 9H) 1.24-1.28 (m, 10H) 8.11 (d, J=2.75 Hz, 2H) 9.05 (s, 1H) 9.88 (s, 1H) 10.16 (s, 1H).

### Step 4: Preparation of N,N′-(3-bromo-5-(3-(trifluoromethyl)-1H-1,2,4-triazol-1-yl)pyridine-2,6-diyl)bis(2,2-dimethylpropanamide)

To a stirred solution of N,N′-(3-(3-(trifluoromethyl)-1H-1,2,4-triazol-1-yl)pyridine-2,6-diyl)bis(2,2-dimethylpropanamide) (76.1 g, 184 mmol) in DMF (1 L) at RT was added NBS (34.5 g, 194 mmol). The mixture was heated to 70° C. for 40 mins, then cooled to 22° C. and poured into ice water (3 L) with stirring. After stirring at RT for 30 mins, a precipitate was collected by filtration. The solid was partitioned between EtOAc (500 mL) and H₂O (500 mL), the layers separated and the organic layer washed with brine (2×300 mL), dried over MgSO₄, filtered and evaporated. The residue was triturated with n-heptane, collected by filtration and dried under vacuum to afford the title compound (84.3 g). LCMS: m/z 491/493 [M+H]⁺. ¹H NMR (600 MHz, DMSO-d₆) delta ppm 1.04 (s, 9H) 1.25 (s, 8H) 8.59 (s, 1H) 9.23 (s, 1H) 9.87-9.93 (m, 1H) 10.04 (s, 1H) 10.28 (s, 1H).

### Step 5: Preparation of Ethyl 2,6-dipivalamido-5-(3-(trifluoromethyl)-1H-1,2,4-triazol-1-yl)nicotinate

A stirred solution of EtOH (152 mL), PhMe (1.1 L) and Et₃N (443 mL, 3176 mmol) was degassed with N₂ for 1 hour. To this was added N,N′-(3-bromo-5-(3-(trifluoromethyl)-1H-1,2,4-triazol-1-yl)pyridine-2,6-diyl)bis(2,2-dimethylpropanamide) (85.0 g, 173 mmol) and PdCl₂(dppf).DCM (28.3 g, 34.6 mmol). The mixture was sparged with gaseous CO (5 L) and then heated to 80° C. under an atmosphere of CO for 18 h. Celite(Trademark) (180 g) was added to the cooled mixture and stirred at RT for 30 mins. The mixture was filtered through a silica pad washing with EtOAc (2 L) and the filtrate evaporated to afford the title compound (115.8 g). LCMS: m/z 485 [M+H]⁺. ¹H NMR (600 MHz, DMSO-d₆) delta ppm 1.05 (s, 9H) 1.24 (s, 9H) 1.26-1.31 (m, 3H) 4.21-4.28 (m, 2H) 8.45 (s, 1H) 9.17 (s, 1H) 10.59 (s, 1H).

### Step 6: Preparation of 2,6-diamino-5-(3-(trifluoromethyl)-1H-1,2,4-triazol-1-yl)nicotinic acid

To a stirred solution of ethyl 2,6-dipivalamido-5-(3-(trifluoromethyl)-1H-1,2,4-triazol-1-yl)nicotinate (84 g, 174 mmol) in IMS (432 mL) at 80° C. was added 4 M aqueous NaOH solution (867 mL, 3467 mmol). The mixture was heated to reflux for 5 h and the cooled mixture diluted with ice water (1.3 L) with stirring, filtered and evaporated to remove residual IMS. The aqueous mixture was washed with TBME (2×1.5 L) and acidified to pH 5 with c. HCl (50 mL) and a 2 M solution of HCl (300 mL) and stood at RT for 18 h forming a precipitate. The solid was collected by filtration, washed with water (2×300 mL) and Et₂O (2×300 mL) and dried under vacuum to afford the title compound (51 g). LCMS: m/z 289 [M+H]⁺. ¹H NMR (600 MHz, DMSO-d₆) delta ppm 6.60 (br s, 2H) 7.45 (br s, 2H) 7.79 (s, 1H) 8.97 (s, 1H) 12.24 (br s, 1H).

### Step 7: Preparation of (R)-2,6-diamino-5-(3-(trifluoromethyl)-1H-1,2,4-triazol-1-yl)-N-(4-((1,1,1-trifluoropropan-2-yl)oxy)benzyl)nicotinamide

To a stirred solution of 2,6-diamino-5-(3-(trifluoromethyl)-1H-1,2,4-triazol-1-yl)nicotinic acid (44.6 g, 155 mmol) and TBTU (52.2 g, 163 mmol) in DMF (650 mL) was added DIPEA (108 mL, 619 mmol) at RT. The mixture was stirred at RT for 15 mins. (R)-(4-((1,1,1-trifluoropropan-2-yl)oxy)phenyl)methanamine hydrochloride (41.5 g, 163 mmol) was added and the mixture stirred at RT for 1 h. The mixture was diluted with EtOAc (1.2 L) and half-saturated brine solution (1 L), and the organic layer washed with water (500 mL), diluted with brine (500 mL) and filtered through Celite(Trademark) with EtOAc (1 L). The organic layer was washed with brine (500 mL), dried over MgSO₄, filtered and evaporated. The residue was suspended in 20% EtOH in n-heptane (800 mL) and heated to reflux for 30 mins. The cooled mixture was filtered, the solid washed with 20% EtOH/n-heptane (2×350 mL) and pentane (2×400 mL), then re-dissolved in EtOAc (500 mL). The experiment above was repeated a total of three times and the combined products were filtered through KP-NH silica (500 g) and the pad washed with EtOAc (7 L). The filtrate was evaporated and the residue triturated with pentane, collected by filtration and dried under vacuum to afford the title compound (50 g). LCMS: m/z 490 [M+H]⁺. ¹H NMR (600 MHz, DMSO-d₆) delta ppm 1.39 (d, J=6.24 Hz, 3H) 4.31 (d, J=5.69 Hz, 2H) 5.15 (app spt, J=6.42 Hz, 1H) 6.39 (s, 2H) 7.01 (d, J=8.25 Hz, 2H) 7.23 (d, J=8.44 Hz, 2H) 7.42 (br s, 2H) 7.92 (s, 1H) 8.41 (br t, J=5.69 Hz, 1H) 8.97 (s, 1H). Chiral HPLC: R, 20.3 mins (minor), 22.9 mins (major), 96.6% ee.

### Preparation of (R)-2,6-diamino-5-(1-(difluoromethyl)-1H-pyrazol-4-yl)-N-(4-((1,1,1-trifluoropropan-2-yl)oxy)benzyl)nicotinamide (II)

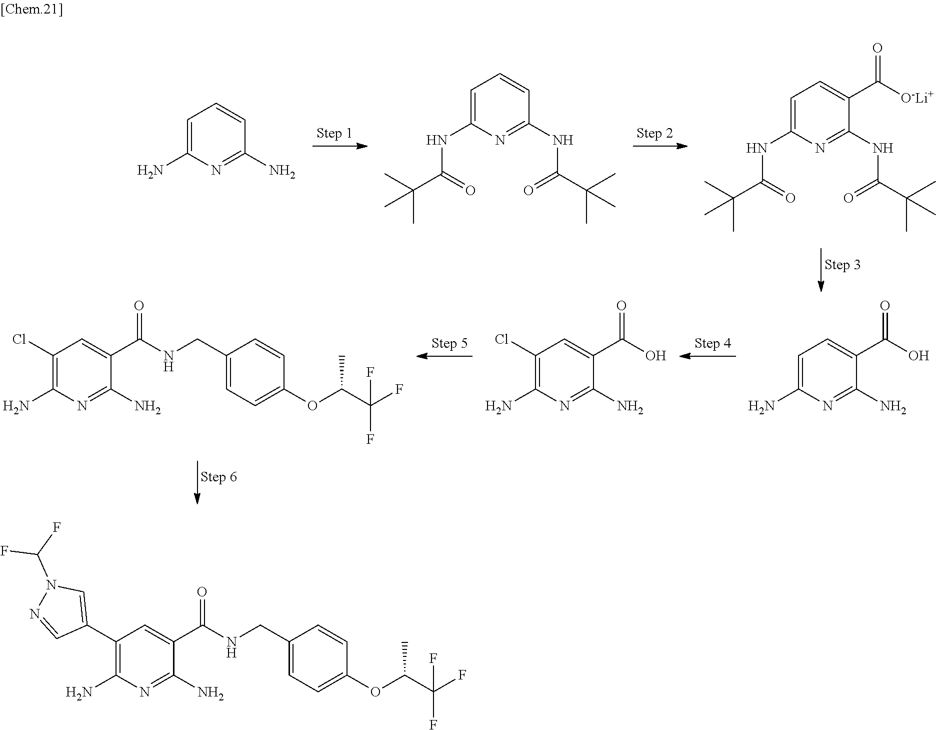

### Step 1: Preparation of N,N′-(pyridine-2,6-diyl)bis(2,2-dimethylpropanamide)

To a stirred suspension of 2,6-diaminopyridine (52 g, 476 mmol) (CAS 141-86-6) in DCM (500 mL) under N₂ at RT was added Et₃N (166 mL, 1191 mmol). The resulting solution was cooled to −2° C. and pivaloyl chloride (129 mL, 1048 mmol) was added dropwise over 1 hour. The resulting solution was allowed to warm slowly to RT overnight then quenched with half-saturated aqueous NaHCO₃ solution (500 mL) and stirred for 20 mins at RT. The aqueous phase was extracted with DCM (100 mL) and the combined organic extracts were dried over Na₂SO₄, filtered and evaporated. The resulting solid was stirred in n-heptane (1200 mL) at reflux for 45 mins, filtered while hot through a warm sinter and the filtrate allowed to cool slowly to RT overnight. The solid was collected by filtration, washed with n-heptane (100 mL) and dried under vacuum to afford the title compound (118 g). LCMS: m/z 278 [M+H]⁺. ¹H NMR (600 MHz, CDCl₃) delta ppm 1.32 (s, 18H), 7.66-7.71 (t, J=8.07 Hz, 1H), 7.74 (br s, 2H), 7.92 (d, J=8.07 Hz, 2H).

### Step 2: Preparation of lithium 2,6-dipivalamidonicotinate

To a stirred solution of N,N-(pyridine-2,6-diyl)bis(2,2-dimethylpropanamide) (59.5 g, 215 mmol) in THF (595 mL) under N₂ at −40° C. was added n-BuLi (2.5 M in hexanes, 300 mL, 750 mmol) dropwise over 80 mins. The mixture was allowed to warm to 0° C. and stirred for 6 h. The mixture was cooled to −30° C. and solid CO₂ pellets (135 g) were added portionwise over 15 mins. The mixture was allowed to warm slowly to RT and stirred for 40 h. The mixture was evaporated and the resulting solid washed with water (2×150 mL) and EtOAc (200 mL), then dried under vacuum to afford the title compound (67.1 g). LCMS: m/z 322 [M+H]⁺. ¹H NMR (600 MHz, DMSO-d) delta ppm 1.21 (s, 9H), 1.24 (s, 9H), 7.59 (d, J=8.44 Hz, 1H), 8.17 (d, J=8.44 Hz, 1H), 9.19 (br s, 1H), 14.48 (br s, 1H).

### Step 3: Preparation of 2,6-diaminonicotinic acid

To lithium 2,6-dipivalamidonicotinate (67.1 g, 205 mmol) was added 4 M aqueous NaOH (360 mL) and the mixture was stirred at 100° C. for 90 mins. The mixture was cooled to 0° C. and c. HCl (118 mL, 1439 mmol) was added dropwise. The mixture was then further acidified to pH 4 with c. HCl, forming a precipitate which was collected by filtration, washed with water (100 mL) and Et₂O (100 mL) and dried under vacuum to afford the title compound (26.4 g). LCMS: m/z 154 [M+H]⁺. ¹H NMR (600 MHz, DMSO-dₜ) delta ppm 5.70 (d, J=8.62 Hz, 1H) 6.33 (br s, 2H) 6.65-7.05 (m, 2H) 7.62 (d, J=8.59 Hz, 1H) 11.61-11.79 (m, 1H).

### Step 4: Preparation of 2,6-diamino-5-chloronicotinic acid

To a slurry of 2,6-diaminonicotinic acid (1.50 g, 9.80 mmol) in DMF (20 mL) was added NCS (1.50 g, 11.3 mmol) and the mixture was stirred at 50° C. for 2 h. The cooled mixture was partitioned between EtOAc and 1 M aqueous NaOH, the layers separated and the organic layer extracted with 1 M NaOH (100 mL). The aqueous extracts were acidified to pH 5 with c. HCl, forming a precipitate which was collected via filtration, washed with water (50 mL) then EtOAc (50 mL) and dried under vacuum to afford the title compound (1.29 g). LCMS: m/z 188/190 [M+H]⁺. ¹H NMR (600 MHz, DMSO-d₆) delta ppm 6.66 (br s, 2H) 6.97 (br s, 2H) 7.67 (s, 1H) 12.13 (br s, 1H).

### Step 5: Preparation of (R)-2,6-diamino-5-chloro-N-(4-((1,1,1-trifluoropropan-2-yl)oxy)benzyl)nicotinamide

To a stirred solution of 2,6-diamino-5-chloronicotinic acid (1.24 g, 6.62 mmol) in DMSO (15 mL) was added HOBT (1.32 g, 8.60 mmol) and EDC.HCl (1.65 g, 8.60 mmol) at RT, followed by a solution of (R)-(4-((1,1,1-trifluoropropan-2-yl)oxy)phenyl)methanamine (1.45 g, 6.62 mmol) in DMSO (15 mL) and Et₃N (3.69 mL, 26.5 mmol). The mixture was stirred at RT for 65 h then partitioned between EtOAc (50 mL) and water (50 mL), the layers separated and the organic layer washed with brine (50 mL), dried over Na₂SO₄ and evaporated. The residue was purified by silica gel chromatography (0-80% EtOAc in DCM) to afford the title compound (2.9 g). LCMS: m/z 389 [M+H]⁺. ¹H NMR (600 MHz, DMSO-d₆) delta ppm 1.39 (d, J=6.42 Hz, 3H) 4.31 (d, J=5.87 Hz, 2H) 5.16 (app spt, J=6.48 Hz, 1H) 6.37 (br s, 2H) 7.02 (d, J=7.80 Hz, 2H) 7.06 (br s, 2H) 7.24 (d, J=8.62 Hz, 2H) 7.88 (s, 1H) 8.48 (br t, J=5.78 Hz, 1H)

### Step 6: Preparation of

### (R)-2,6-diamino-5-(1-(difluoromethyl)-1H-pyrazol-4-yl)-N-(4-((1,1,1-trifluoropropan-2-yl)oxy)benzyl)nicotinamide (11)

A mixture of (R)-2,6-diamino-5-chloro-N-(4-((1,1,1-trifluoropropan-2-yl)oxy)benzyl)nicotinamide (150 mg, 0.386 mmol), 1-(difluoromethyl)-4-(4,4,5,5-tetramethyl-1,3,2-dioxaborolan-2-yl)-1H-pyrazole (188 mg, 0.772 mmol) (CAS 1206640-82-5), Pd(OAc)₂ (8.66 mg, 0.039 mmol), SPhos (23.8 mg, 0.058 mmol) and K₃PO₄ (246 mg, 1.16 mmol) in MeCN (9 mL) and H₂O (3 mL) was degassed with N₂ and heated to 90° C. in a sealed tube for 18 h. The cooled mixture was purified by Isolute(Trademark) SCX-2 cartridge and by silica gel chromatography (20-100% EtOAc in cyclohexane) then triturated with DCM to afford the title compound (77 mg). LCMS: m/z 471 [M+H]⁺. ¹H NMR (600 MHz, DMSO-d₆) delta ppm 1.39 (d, J=6.42 Hz, 3H) 4.35 (d, J=5.69 Hz, 2H) 5.16 (app spt, J=6.79 Hz, 1H) 5.95 (s, 2H) 7.02 (d, J=8.62 Hz, 2H) 7.05 (br s, 2H) 7.25 (d, J=8.44 Hz, 2H) 7.79 (t, J=59.05 Hz, 1H) 7.86 (s, 1H) 7.98 (s, 1H) 8.31 (s, 1H) 8.47 (t, J=5.87 Hz, 1H).

### Preparation of (R)-2,6-diamino-5-(1-(difluoromethyl)-3-methyl-1H-pyrazol-4-yl)-N-(4-((1,1,1-trifluoropropan-2-yl)oxy)benzyl)nicotinamide (III)

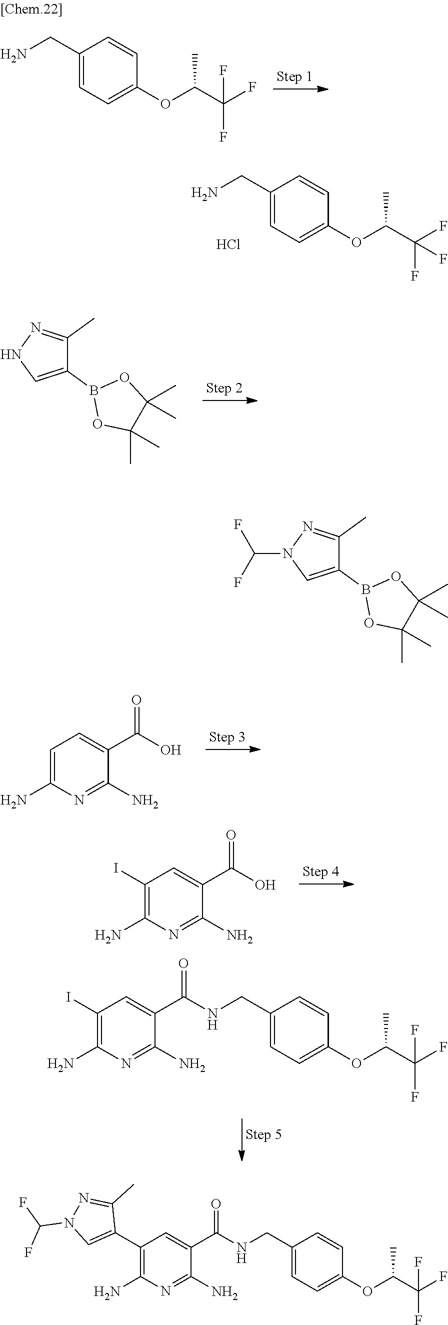

### Step 1: Preparation of (K)-(4-((1,1,1-trifluoropropan-2-yl)oxy)phenyl)methanamine hydrochloride

To a stirred solution of (R)-(4-((1,1,1-trifluoropropan-2-yl)oxy)phenyl)methanamine (4.9 g, 22.4 mmol) in Et₂O (10 mL) at 0° C. was added a 4 N solution of HCl in 1,4-dioxane (6.15 mL, 24.6 mmol). The mixture was allowed to warm to RT over 30 mins forming a precipitate which was collected by filtration, washed with Et₂O (100 mL) and dried under vacuum to afford the title compound (5.23 g). LCMS: m/z 203 [M-NH₂]⁺. ¹H NMR (600 MHz, DMSO-d₆) delta ppm 1.41 (d, J=6.42 Hz, 3H) 3.96 (s, 2H) 5.27 (app spt, J=6.45 Hz, 1H) 7.13 (d, J=8.62 Hz, 2H) 7.44 (d, J=8.62 Hz, 2H) 8.24 (br s, 3H).

### Step 2: Preparation of 1-(difluoromethyl)-3-methyl-4-(4,4,5,5-tetramethyl-1,3,2-dioxaborolan-2-yl)-1H-pyrazole

A mixture of 3-methyl-4-(4,4,5,5-tetramethyl-1,3,2-dioxaborolan-2-yl)-1H-pyrazole (250 mg, 1.20 mmol) (CAS 936250-20-3, Fluorochem), sodium chlorodifluoroacetate (220 mg, 1.44 mmol) and 18-crown-6 (63.5 mg, 0.240 mmol) in MeCN (8 mL) was heated to 80° C. under N₂ for 18 h. The mixture was evaporated, the residue partitioned between EtOAc and sat. aqueous NH₄Cl, the layers separated and the organic layer dried over Na₂SO₄ and evaporated. The residue was purified by silica gel chromatography (0-50% EtOAc in cyclohexane) to afford the title compound (212 mg) as the major component of a 3:1 mixture of regioisomers. LCMS: m/z 259 [M+H]⁺. ¹H NMR (600 MHz, CDCl₃) delta ppm 1.31 (s, 12H) 2.41 (s, 3H) 7.12 (t, J=60.9 Hz, 1H) 8.02 (s, 1H).

### Step 3: Preparation of 2,6-diamino-5-iodonicotinic acid

To a stirred suspension of 2,6-diaminonicotinic acid (500 mg, 3.27 mmol) in DMF (10 mL) at RT was added NIS (808 mg, 3.59 mmol) portionwise. After 5 mins, the mixture was partitioned between EtOAc and 1 M aqueous NaOH solution and the layers separated. The aqueous layer was acidified to pH 5 with c. HCl forming a precipitate which was collected by filtration, washed with H₂O (10 mL) and EtOAc (10 mL) and dried under vacuum to afford the title compound (776 mg). LCMS: m/z 280 [M+H]⁺. ¹H NMR (600 MHz, DMSO-d₆) delta ppm 6.37 (br s, 2H) 6.94 (br s, 2H) 7.99 (s, 1H) 12.13 (br s, 1H).

### Step 4: Preparation of (R)-2,6-diamino-5-iodo-N-(4-((1,1,1-trifluoropropan-2-yl)oxy)benzyl)nicotinamide

To a stirred solution of 2,6-diamino-5-iodonicotinic acid (200 mg, 0.717 mmol) and TBTU (276 mg, 0.860 mmol) in DMF (4 mL) at RT was added DIPEA (501 micro L, 2.87 mmol). The mixture was stirred for 30 mins at RT, (R)-(4-((1,1,1-trifluoropropan-2-yl)oxy)phenyl)methanamine hydrochloride (220 mg, 0.860 mmol) was added and the mixture stirred for 2 h at RT. H₂O (4 mL) was added and the mixture stirred at RT for 5 mins, then diluted with EtOAc (20 mL) and the layers separated. The organic layer was washed with water (3×10 mL) and brine (2×10 mL), dried over Na₂SO₄, filtered and evaporated. The residue was triturated with 20% Et₂O in n-pentane (10 mL) to afford the title compound (276 mg). LCMS: m/z 481 [M+H]⁺. ¹H NMR (600 MHz, DMSO-ds) delta ppm 1.40 (d, J=6.24 Hz, 3H) 4.30 (d, J=5.69 Hz, 2H) 5.16 (dt, J=12.84, 6.42 Hz, 1H) 6.11 (br s, 2H) 7.02 (d, J=8.25 Hz, 2H) 7.06 (br s, 2H) 7.23 (d, J=8.44 Hz, 2H) 8.11 (s, 1H) 8.50 (t, J=5.69 Hz, 1H).

### Step 5: Preparation of (R)-2,6-diamino-5-(1-(difluoromethyl)-3-methyl-1H-pyrazol-4-yl)-N-(4-((1,1,1-trifluoropropan-2-yl)oxy)benzyl)nicotinamide

A mixture of (R)-2,6-diamino-5-iodo-N-(4-((1,1,1-trifluoropropan-2-yl)oxy)benzyl)nicotinamide (50 mg, 0.104 mmol), 1-(difluoromethyl)-3-methyl-4-(4,4,5,5-tetramethyl-1,3,2-dioxaborolan-2-yl)-1H-pyrazole (55 mg, 0.213 mmol), Pd(OAc)₂ (2.34 mg, 10.4 micro mol), SPhos (6.41 mg, 0.016 mmol) and K₃PO₄ (66.3 mg, 0.312 mmol) in MeCN (1.5 mL) and H₂O (0.5 mL) was degassed with N₂ and heated to 100° C. in a sealed tube for 20 h. The cooled mixture was purified by Isolute(Trademark) SCX-2 cartridge, by silica gel chromatography (20-100% EtOAc in cyclohexane) and then by MDAP to afford the title compound (6 mg). LCMS: m/z 485 [M+H]⁺. ¹H NMR (600 MHz, DMSO-d_{b}) delta ppm 1.38 (d, J=6.42 Hz, 3H) 2.13 (s, 3H) 4.30 (d, J=5.87 Hz, 2H) 5.15 (app spt, J=6.45 Hz, 1H) 5.80 (s, 2H) 7.01 (d, J=8.62 Hz, 2H) 7.06 (br s, 2H) 7.22 (d, J=8.44 Hz, 2H) 7.63 (s, 1H) 7.69 (t, J=59.6 Hz, 1H) 8.07 (s, 1H) 8.37 (t, J=5.96 Hz, 1H).

### Preparation of 2,6-diamino-N-(4-(2,2,2-trifluoroethoxy)benzyl)-5-(3-(trifluoromethyl)-1H-1,2,4-triazol-1-yl)nicotinamide (IV)

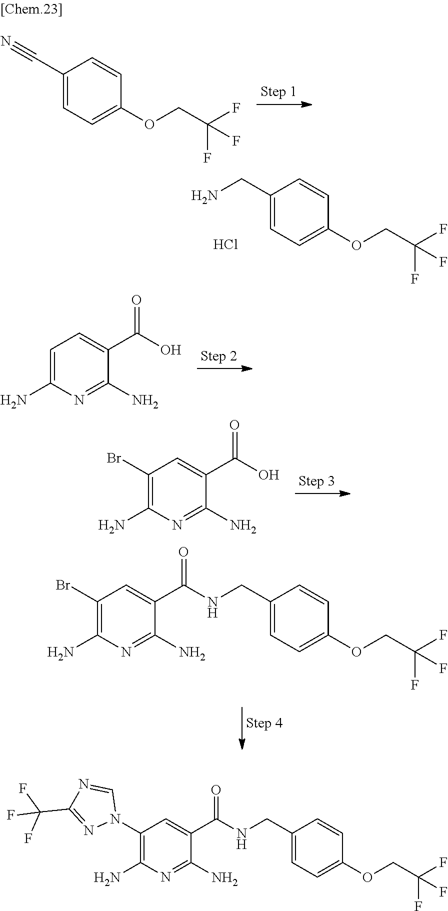

### Step 1: Preparation of (4-(2,2,2-trifluoroethoxy)phenyl)methanamine hydrochloride

To a stirred solution of 4-(2,2,2-trifluoroethoxy)benzonitrile (6.43 g, 31.9 mmol) (CAS 56935-76-3, Combi-Blocks) in THF (64 mL) under N₂ at 0° C. was added BH₃.THF (1 M in THF, 63.9 mL, 63.9 mmol). The mixture was stirred at 0° C. for 30 mins then heated to reflux for 16 h. To the cooled mixture was added a 2 M HCl (63.9 mL, 128 mmol). The mixture was heated to 100° C. for 2 h. The mixture was evaporated and the residue washed with water (3×20 mL) and Et₃O (3×20 mL) to afford the title compound (7.32 g). LCMS: m/z 190 [M-NH₂]⁺. ¹H NMR (600 MHz, DMSO-d₆) delta ppm 3.96 (q, J=5.62 Hz, 2H) 4.79 (q, J=8.99 Hz, 2H) 7.10 (d, J=8.62 Hz, 2H) 7.46 (d, J=8.62 Hz, 2H) 8.31 (br s, 3H).

### Step 2: Preparation of 2,6-diamino-5-bromonicotinic acid

To a suspension of finely powdered 2,6-diaminonicotinic acid (5.69 g, 37.2 mmol) in AcOH (31 mL) was slowly added a solution of Br, (2.20 mL, 42.7 mmol) in AcOH (16 mL) at RT. The mixture was stirred for 30 mins to form a precipitate which was collected by filtration, washed with AcOH (2×30 mL), H₂O (2×30 mL), Et₂O (30 mL) and dried under vacuum to afford the title compound (8.44 g). LCMS: m/z 232/234 [M+H]⁺. ¹H NMR (600 MHz, DMSO-d₆) delta ppm 7.37 (br s, 4H) 7.99 (s, 1 H). CO₂H not observed.

### Step 3: Preparation of 2,6-diamino-5-bromo-N-(4-(2,2,2-trifluoroethoxy)benzyl)nicotinamide

To a stirred solution of 2,6-diamino-5-bromonicotinic acid (4 g, 17.2 mmol) in DMSO (60 mL) at RT was added Et₃N (9.61 mL, 69.0 mmol), EDC.HCl (4.30 g, 22.4 mmol) and HOBt (3.43 g, 22.4 mmol). The mixture was stirred at RT for 30 mins. (4-(2,2,2-trifluoroethoxy)phenyl)methanamine hydrochloride (5.42 g, 22.4 mmol) was added and the mixture stirred at RT for 16 h. The mixture was diluted with H₂O (250 mL) and stirred for 1 h, forming a precipitate which was collected by filtration, washed with H₂O (50 mL) dried under vacuum, and then recrystallized from EtOAc to afford the title compound (5.9 g). LCMS: m/z 419/421 [M+H]⁺. ¹H NMR (600 MHz, DMSO-d₆) delta ppm 4.31 (d, J=5.87 Hz, 2H) 4.72 (q, J=8.99 Hz, 2H) 6.30 (br s, 2H) 7.00 (d, J=7.94 Hz, 2H) 7.08 (br s, 2H) 7.24 (d, J=8.62 Hz, 2H) 8.00 (s, 1H) 8.51 (t, J=5.78 Hz, 1H).

### Step 4: Preparation of 2,6-diamino-N-(4-(2,2,2-trifluoroethoxy)benzyl)-5-(3-(trifluoromethyl)-1H-1,2,4-triazol-1-yl)nicotinamide

A mixture of 2,6-diamino-5-bromo-N-(4-(2,2,2-trifluoroethoxy)benzyl)nicotinamide (30 mg, 0.07 mmol), 3-(trifluoromethyl)-1H-1,2,4-triazole (13.73 mg, 0.10 mmol) (CAS 60406-75-9, Enamine), copper(I) iodide (15.0 mg, 0.079 mmol), (R,R)-(−)-N,N′-dimethyl-1,2-cyclohexanediamine (12.41 micro L, 0.079 mmol) and K₃PO₄ (30.4 mg, 0.143 mmol) in DMF (0.8 mL) was degassed with N₂ and heated to 120° C. in a sealed tube for 18 h. The cooled mixture was purified by Isolute(Trademark) SCX-2 cartridge and by silica gel chromatography (20-100% EtOAc in cyclohexane) to afford the title compound (5 mg). LCMS: m/z 476 [M+H]⁺. ¹H NMR (600 MHz, DMSO-d₆) delta ppm 4.31 (d, J=5.69 Hz, 2H) 4.71 (q, J=8.93 Hz, 2H) 6.40 (s, 2H) 6.99 (d, J=8.62 Hz, 2H) 7.24 (d, J=8.62 Hz, 2H) 7.43 (br s, 2H) 7.92 (s, 1H) 8.42 (t, J=5.78 Hz, 1H) 8.98 (s, 1H).

### Preparation of (R)-2,6-diamino-5-(3-(difluoromethyl)-1H-1,2,4-triazol-1-yl)-N-(4-((1,1,1-trifluoropropan-2-yl)oxy)benzyl)nicotinamide (V)

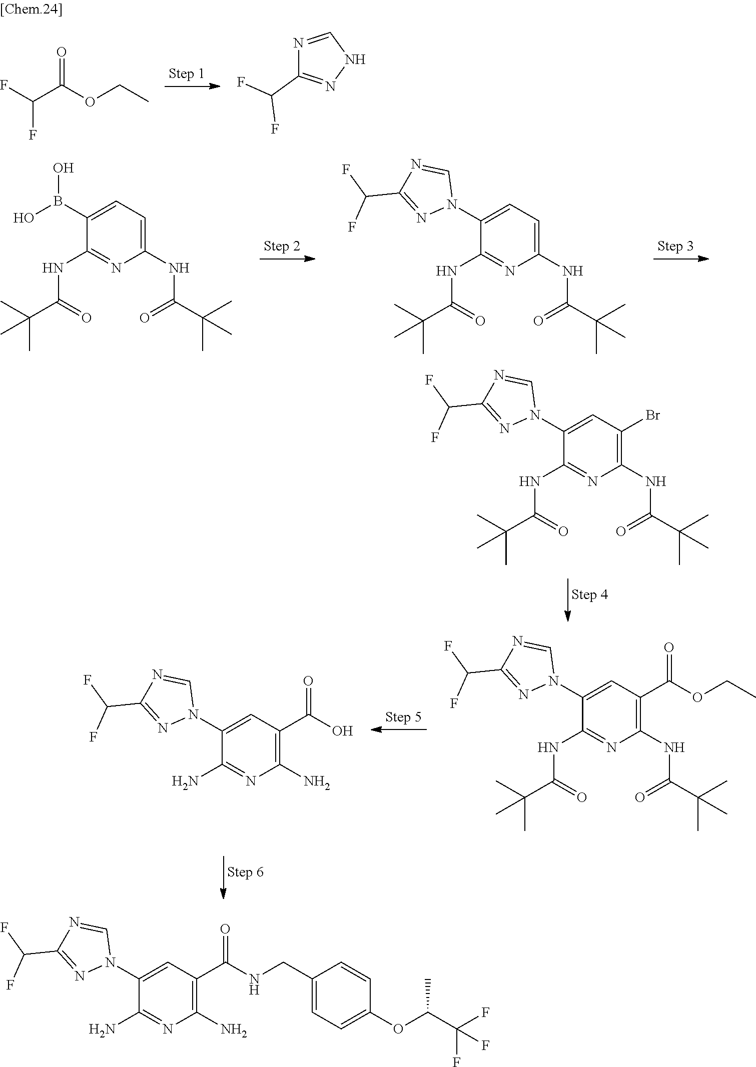

### Step 1: Preparation of 3-(difluoromethyl)-1H-1,2,4-triazole

To a stirred solution of hydrazine hydrate (18.6 mL, 383 mmol) in EtOH (500 mL) at 0° C. was added ethyl difluoroacetate (42.4 mL, 403 mmol) (CAS 454-31-9) dropwise. The mixture was stirred at RT for 16 h. Iminoformamide acetate (46.1 g, 443 mmol) was added and the mixture heated to reflux for 5 h. To the cooled mixture was added AcOH (34.6 mL, 604 mmol) and the EtOH removed by evaporation. The mixture was diluted with water (200 mL) and basified to pH 8 with solid Na₂CO₃. The aqueous solution was extracted with EtOAc (3×250 mL), the combined organic extracts dried over Na₂SO₄, filtered and evaporated. The residue was triturated with 1:1 TBME:n-heptane and 9:1 TBME:n-heptane successively, the solid collected by filtration and dried under vacuum to afford the title compound (30.5 g). ¹H NMR (600 MHz, DMSO-d₆) delta ppm 7.08 (t, J=53.19 Hz, 1H) 8.70 (s, 1H) 14.51 (br s, 1H).

### Step 2: Preparation of N,N′-(3-(3-(difluoromethyl)-1H-1,2,4-triazol-1-yl)pyridine-2,6-diyl)bis(2,2-dimethylpropanamide)

To a stirred solution of (2,6-dipivalamidopyridin-3-yl)boronic acid (500 mg, 1.56 mmol) in DMF (600 mL) was added 3-(difluoromethyl)-1H-1,2,4-triazole (204 mg, 1.71 mmol), copper(II) acetate (28.3 mg, 0.156 mmol) and pyridine (0.315 mL, 3.89 mmol). A low vacuum was placed on the three-neck flask allowing air to pass through a drying tube containing anhydrous CaCl₂ and into the solution through a sparging tube. The mixture was heated to 60° C. for 16 h then cooled and poured into ice water (30 mL). After 30 mins a precipitate was collected by filtration, washed with H₂O (20 mL) and dried under vacuum to afford the title compound (501 mg). LCMS: m/z 395 [M+H]⁺. ¹H NMR (600 MHz, DMSO-d₆) delta ppm 1.03 (s, 9H) 1.26 (s, 9H) 7.12 (t, J=53.00 Hz, 1H) 8.07 (d, J=8.44 Hz, 1H) 8.09 (d, J=8.62 Hz, 1H) 8.86 (s, 1H) 9.81 (s, 1H) 10.14 (s, 1H).

### Step 3: Preparation of N,N′-(3-bromo-5-(3-(difluoromethyl)-1H-1,2,4-triazol-1-yl)pyridine-2,6-diyl)bis(2,2-dimethylpropanamide)

To a stirred solution of N,N′-(3-(3-(difluoromethyl)-1H-1,2,4-triazol-1-yl)pyridine-2,6-diyl)bis(2,2-dimethylpropanamide) (3.13 g, 7.94 mmol) in DMF (58 mL) was added NBS (1.41 g, 7.94 mmol) at RT. The mixture was heated to 70° C. for 20 mins and the cooled mixture poured into ice water (120 mL) and stirred for 5 mins, forming a precipitate which was collected by filtration, washed with H₂O (30 mL) and dried under vacuum to afford the title compound (3.57 g). LCMS: m/z 473/475 [M+H]⁺. ¹H NMR (600 MHz, DMSO-d₆) delta ppm 1.04 (s, 9H) 1.25 (s, 9H) 7.13 (t, J=52.45 Hz, 1H) 8.53 (s, 1H) 9.04 (s, 1H) 10.01 (s, 1H) 10.20 (s, 1H).

### Step 4: Preparation of ethyl 5-(3-(difluoromethyl)-1H-1,2,4-triazol-1-yl)-2,6-dipivalamidonicotinate

A stirred solution of EtOH (14 mL), PhMe (113 mL) and Et₃N (45.3 mL, 325 mmol) was degassed with N₂ for 1 hour. To this was added N,N′-(3-bromo-5-(3-(difluoromethyl)-1H-1,2,4-triazol-1-yl)pyridine-2,6-diyl)bis(2,2-dimethylpropanamide) (7.55 g, 16.0 mmol) and PdCl₂(dppf).DCM (2.61 g, 3.19 mmol). The mixture was sparged with gaseous CO (2 L) and then heated to 80° C. under an atmosphere of CO for 21 h. Celite(Trademark) (10 g) was added to the cooled mixture and the mixture was filtered through a silica pad washing with EtOAc (500 mL) and the filtrate evaporated to afford the title compound (8.89 g). LCMS: m/z 467 [M+H]⁺. ¹H NMR (600 MHz, DMSO-d₆) delta ppm 1.05 (s, 9H) 1.24 (s, 9H) 1.28 (t, J=7.15 Hz, 3H) 4.24 (q, J=7.03 Hz, 2H) 7.15 (t, J=52.64 Hz, 2H) 8.38 (s, 1H) 9.00 (s, 1H) 10.26 (s, 1H) 10.56 (s, 1H).

### Step 5: Preparation of 2,6-diamino-5-(3-(difluoromethyl)-1H-1,2,4-triazol-1-yl)nicotinic acid

To a stirred solution of ethyl 5-(3-(difluoromethyl)-1H-1,2,4-triazol-1-yl)-2,6-dipivalamidonicotinate (7.44 g, 16.0 mmol) in IMS (40 mL) at 80° C. was added 4 M aqueous NaOH (80 mL, 319 mmol). The mixture was heated to reflux for 3 h then cooled and diluted with H₂O (120 mL) with stirring, filtered and evaporated to remove residual IMS. The aqueous mixture was washed with TBME (2×50 mL), acidified to pH 4 with c. HCl and stirred for 5 mins forming a precipitate. The solid was collected by filtration, washed with H₂O (50 mL) and Et₂O (50 mL) and dried under vacuum to afford the title compound (3.50 g). LCMS: m/z 271 [M+H]⁺. ¹H NMR (600 MHz, DMSO-d₆) delta ppm 6.56 (br s, 2H) 7.11 (t, J=53.19 Hz, 1H) 7.25 (br s, 2H) 7.76 (s, 1H) 8.87 (s, 1H) 12.24 (br s, 1H).

### Step 6: Preparation of (R)-2,6-diamino-5-(3-(difluoromethyl)-1H-1,2,4-triazol-1-yl)-N-(4-((1,1,1-trifluoropropan-2-yl)oxy)benzyl)nicotinamide

To a stirred solution of 2,6-diamino-5-(3-(difluoromethyl)-1H-1,2,4-triazol-1-yl)nicotinic acid (3.35 g, 12.4 mmol) and TBTU (4.18 g, 13.0 mmol) in DMF (45 mL) was added DIPEA (8.65 mL, 49.5 mmol) at RT. The mixture was stirred at RT for 15 mins a solution of (R)-(4-((1,1,1-trifluoropropan-2-yl)oxy)phenyl)methanamine (3.12 g, 14.2 mmol) in DMF (5 mL) was added and the mixture stirred at RT for 40 mins. The mixture was diluted with EtOAc (100 mL), washed with H₂O (150 mL), brine (150 mL), H₂O (200 mL) and brine (200 mL), dried by passing through hydrophobic filter paper and evaporated. The residue was recrystallized from 1:1 EtOH:n-heptane (29 mL) forming a solid which was collected by filtration, washed with 9:1 n-heptane:EtOH (50 mL) and dried under vacuum to afford the title compound (4.70 g). LCMS: m/z 472 [M+H]⁺. ¹H NMR (600 MHz, DMSO-d₆) delta ppm 1.39 (d, J=6.24 Hz, 3H) 4.31 (d, J=5.69 Hz, 2H) 5.15 (app spt, J=6.45 Hz, 1H) 6.33 (s, 2H) 7.01 (d, J=8.62 Hz, 2H) 7.12 (t, J=53.19 Hz, 1H) 7.23 (d, J=8.62 Hz, 2H) 7.26-7.55 (m, 2H) 7.92 (s, 1H) 8.41 (t, J=5.69 Hz, 1H) 8.85 (s, 1H). Chiral HPLC: R, 30.8 mins (minor), 34.5 (major), 97.3% ee.

### Preparation of (R)-2,6-diamino-5-(3-(2,2,2-trifluoroethyl)-1H-1,2,4-triazol-1-yl)-N-(4-((1,1,1-trifluoropropan-2-yl)oxy)benzyl)nicotinamide (VI)

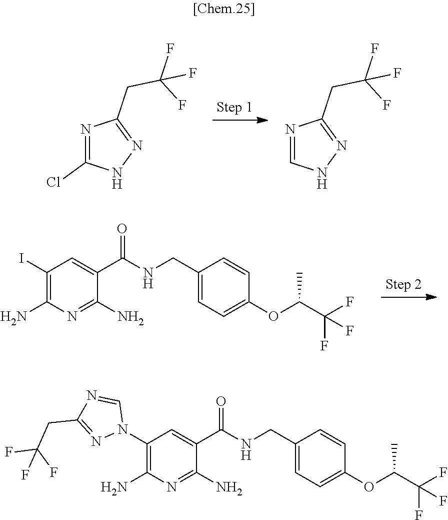 [SMILES: FC(F)(F)Cc1n[nH]c(Cl)n1 ; FC(F)(F)Cc1nc[nH]n1 ; C[C@@H](Oc1ccc(CNC(=O)c2cc(I)c(N)nc2N)cc1)C(F)(F)F ; C[C@@H](Oc1ccc(CNC(=O)c2cc(-n3cnc(CC(F)(F)F)n3)c(N)nc2N)cc1)C(F)(F)F]

### Step 1: Preparation of 3-(2,2,2-trifluoroethyl)-1H-1,2,4-triazole

A stirred solution of 5-chloro-3-(2,2,2-trifluoroethyl)-1H-1,2,4-triazole (120 mg, 0.647 mmol) (CAS 1340078-75-2) in IMS (3 mL) was degassed with N₂. To this was added Pd(OH)₂ (42 mg, 0.299 mmol), the reaction vessel was evacuated and backfilled with H₂ three times, then the mixture was stirred under an atmosphere of H₂ at RT for 65 h. The mixture was filtered through Celite(Trademark), washing with IMS (30 mL). The filtrate was purified by Isolute(Trademark) SCX-2 cartridge to afford the title compound (55 mg). LCMS: m/z 152 [M+H]⁺. ¹H NMR (600 MHz, DMSO-d₆) delta ppm 3.78 (br q, J=10.45 Hz, 2H) 8.47 (br s, 1H) 14.05 (br s, 1H).

### Step 2: Preparation of (R)-2,6-diamino-5-(3-(2,2,2-trifluoroethyl)-1H-1,2,4-triazol-1-yl)-N-(4-((1,1,1-trifluoropropan-2-yl)oxy)benzyl)nicotinamide

A mixture of (R)-2,6-diamino-5-iodo-N-(4-((1,1,1-trifluoropropan-2-yl)oxy)benzyl)nicotinamide (50 mg, 0.104 mmol), 3-(2,2,2-trifluoroethyl)-1H-1,2,4-triazole (20.5 mg, 0.135 mmol), copper(I) iodide (21.8 mg, 0.115 mmol), (R,R)-(−)-N,N′-dimethyl-1,2-cyclohexanediamine (18.1 micro L, 0.115 mmol) and K₃PO₄ (44.2 mg, 0.208 mmol) in DMF (0.8 mL) was degassed with N₂ and heated to 120° C. in a sealed tube for 18 h. The cooled mixture was purified by Isolute(Trademark) SCX-2 cartridge and by MDAP to afford the title compound (15 mg). LCMS: m/z 504 [M+H]⁺. ¹H NMR (600 MHz, DMSO-d₆) delta ppm 1.39 (d, J=6.42 Hz, 3H) 3.83 (q, J=11.13 Hz, 2H) 4.32 (d, J=5.87 Hz, 2H) 5.15 (app spt, J=6.45 Hz, 1H) 6.27 (s, 2H) 7.01 (d, J=8.62 Hz, 2H) 7.24 (d, J=8.44 Hz, 2H) 7.34 (br s, 2H) 7.94 (s, 1H) 8.46 (t, J=5.87 Hz, 1H) 8.72 (s, 1H).

### Preparation of 2,6-diamino-N-(4-(2,2,2-trifluoroethoxy)benzyl)-5-(3-(1-(trifluoromethyl)cyclopropyl)-1H-1,2,4-triazol-1-yl)nicotinamide (VII)

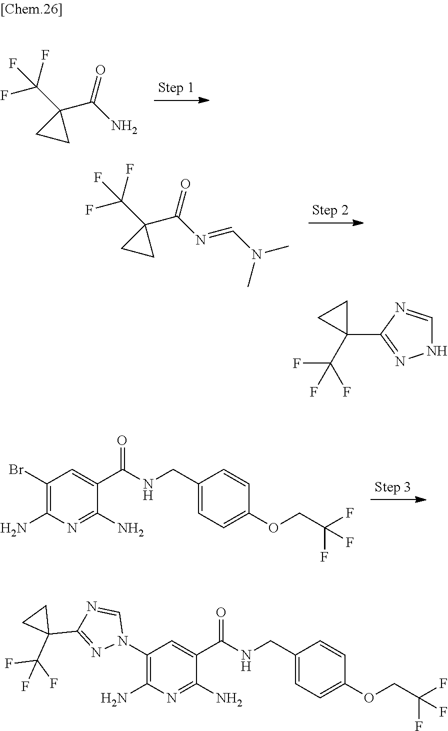 [SMILES: NC(=O)C1(C(F)(F)F)CC1 ; CN(C)/C=N/C(=O)C1(C(F)(F)F)CC1 ; FC(F)(F)C1(c2nc[nH]n2)CC1 ; Nc1nc(N)c(C(=O)NCc2ccc(OCC(F)(F)F)cc2)cc1Br ; Nc1nc(N)c(-n2cnc(C3(C(F)(F)F)CC3)n2)cc1C(=O)NCc1ccc(OCC(F)(F)F)cc1]

### Step 1: Preparation of (E)-N-((dimethylamino)methylene)-1-(trifluoromethyl)cyclopropane-1-carboxamide

A solution of 1-(trifluoromethyl)cyclopropane-1-carboxamide (200 mg, 1.31 mmol) (CAS 1628184-67-7) in DMF-DMA (2.62 mL, 19.6 mmol) was stirred at RT for 1 h. The mixture was evaporated to afford the title compound (272 mg). LCMS: m/z 209 [M+H]⁺. ¹H NMR (600 MHz, CDCl₃) delta ppm 1.26 (q, J=4.03 Hz, 2H) 1.46-1.50 (m, 2H) 3.06 (s, 3H) 3.12 (s, 3H) 8.43 (s, 1H).

### Step 2: Preparation of 3-(1-(trifluoromethyl)cyclopropyl)-1H-1,2,4-triazole

To a stirred solution of (E)-N-((dimethylamino)methylene)-1-(trifluoromethyl)cyclopropane-1-carboxamide (208 mg, 0.999 mmol) in AcOH (4 mL) at 0° C. was added hydrazine hydrate (0.053 mL, 1.10 mmol). The mixture was allowed to warm to RT and stirred for 2 h, then heated to 50° C. for 1.5 h. The mixture was evaporated and the residue triturated with Et₂O to afford the title compound (130 mg). LCMS: m/z 178 [M+H]⁺. ¹H NMR (600 MHz, DMSO-d₆) delta ppm 1.31-1.34 (m, 2H) 1.38-1.42 (m, 2H) 8.45 (br s, 1H) 13.98 (br s, 1H).

### Step 3: Preparation of 2,6-diamino-N-(4-(2,2,2-trifluoroethoxy)benzyl)-5-(3-(1-(trifluoromethyl)cyclopropyl)-1H-1,2,4-triazol-1-yl)nicotinamide

A mixture of 2,6-diamino-5-bromo-N-(4-(2,2,2-trifluoroethoxy)benzyl)nicotinamide (50.0 mg, 0.119 mmol), 3-(1-(trifluoromethyl)cyclopropyl)-1H-1,2,4-triazole (27.5 mg, 0.155 mmol), copper(I) iodide (25.0 mg, 0.131 mmol), K₃PO₄ (76.0 mg, 0.358 mmol) and (R,R)-(−)-N,N′-dimethyl-1,2-cyclohexanediamine (28.2 micro L, 0.179 mmol) in DMF (0.9 mL) was degassed with N₂ and heated to 120° C. in a sealed tube for 16 h. The cooled mixture was purified by Isolute(Trademark) SCX-2 cartridge and by MDAP to afford the title compound (17.0 mg). LCMS: m/z 516 [M+H]⁺. ¹H NMR (600 MHz, DMSO-d₆) delta ppm 1.41-1.44 (m, 4H) 4.32 (d, J=5.69 Hz, 2H) 4.71 (q, J=8.93 Hz, 2H) 6.25 (br s, 2H) 6.99 (d, J=8.62 Hz, 2H) 7.24 (d, J=8.62 Hz, 2H) 7.34 (br s, 2H) 7.91 (s, 1H) 8.44 (t, J=6.05 Hz, 1H) 8.65 (s, 1H)

### Preparation of (R)-2,6-diamino-5-(3-(1-fluorocyclopropyl)-1H-1,2,4-triazol-1-yl)-N-(4-((1,1,1-trifluoropropan-2-yl)oxy)benzyl)nicotinamide (VIII)

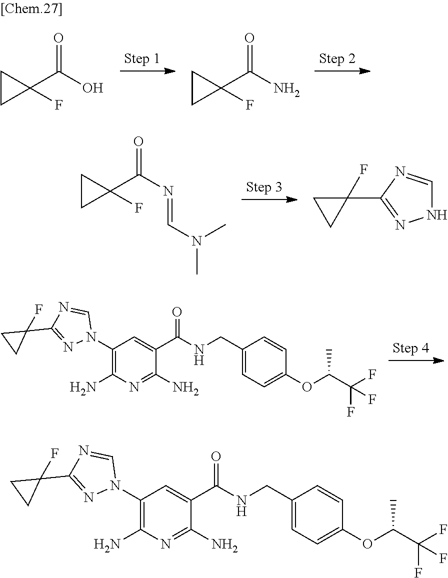 [SMILES: O=C(O)C1(F)CC1 ; NC(=O)C1(F)CC1 ; CN(C)/C=N/C(=O)C1(F)CC1 ; FC1(c2nc[nH]n2)CC1 ; C[C@@H](Oc1ccc(CNC(=O)c2cc(-n3cnc(C4(F)CC4)n3)c(N)nc2N)cc1)C(F)(F)F ; C[C@@H](Oc1ccc(CNC(=O)c2cc(-n3cnc(C4(F)CC4)n3)c(N)nc2N)cc1)C(F)(F)F]

### Step 1: Preparation of 1-fluorocyclopropane-1-carboxamide

To a stirred solution of 1-fluorocyclopropane-1-carboxylic acid (700 mg, 6.73 mmol) (CAS 137081-41-5) in DMF (25 mL) at RT was added NH₄Cl (719 mg, 13.5 mmol), HATU (5.12 g, 13.5 mmol) and DIPEA (3.52 mL, 20.2 mmol). The mixture was stirred at RT for 16 h then partitioned between EtOAc and H₂O, the organic layer washed with a 5% w/v aqueous solution of LiCl (50 mL), brine (50 mL) and dried by passing through hydrophobic filter paper and evaporated. The residue was twice suspended in 20% EtOAc in Et₂O (20 mL), stirred then the supernatant decanted. The solid was triturated with EtOAc and filtered. The combined filtrates were evaporated, the residue dissolved in 1 M HCl (50 mL) and extracted with EtOAc. The organic layer was washed with sat. aqueous NaHCO₃ (50 mL) and brine (50 mL), dried over MgSO₄ and evaporated. The residue was triturated with DCM to afford the title compound (182 mg). LCMS: m/z 104 [M+H]⁺. ¹H NMR (600 MHz, DMSO-d₆) delta ppm 1.10-1.17 (m, 2H) 1.20-1.28 (m, 2H) 7.55 (br s, 1H) 7.76 (br s, 1H).

### Step 2: Preparation of (E)-N-((dimethylamino)methylene)-1-fluorocyclopropane-1-carboxamide

A solution of 1-fluorocyclopropane-1-carboxamide (180 mg, 1.75 mmol) in DMF-DMA (3.5 mL, 26.1 mmol) was stirred at RT for 1 h. The mixture was evaporated to afford the title compound (276 mg). LCMS: m/z 159 [M+H]⁺. ¹H NMR (600 MHz, DMSO-d₆) delta ppm 1.19-1.36 (m, 4H) 2.94 (s, 3H) 3.13 (s, 3H) 8.45 (s, 1H).

### Step 3: Preparation of 5-(1-fluorocyclopropyl)-1H-1,2,4-triazole

To a stirred suspension of (E)-N-((dimethylamino)methylene)-1-fluorocyclopropane-1-carboxamide (276 mg, 1.75 mmol) in AcOH (7 mL) at RT was added hydrazine hydrate (0.093 mL, 1.92 mmol). The mixture was stirred at RT for 2 h, evaporated to 10% volume and the residue basified with a sat. aqueous NaHCO₃, extracted with EtOAc (2×50 mL) and the combined organic extracts washed with brine (1×50 mL), dried by passing through hydrophobic filter paper and evaporated to afford the title compound (82 mg). LCMS: m/z 128 [M+H]⁺. ¹H NMR (600 MHz, DMSO-ds) delta ppm 1.15-1.23 (m, 2H) 1.43-1.54 (m, 2H) 8.36 (br s, 1H) 14.08 (br s, 1H).

### Step 4: Preparation of (R)-2,6-diamino-5-(3-(1-fluorocyclopropyl)-1H-1,2,4-triazol-1-yl)-N-(4-((1,1,1-trifluoropropan-2-yl)oxy)benzyl)nicotinamide

A mixture of (R)-2,6-diamino-5-iodo-N-(4-((1,1,1-trifluoropropan-2-yl)oxy)benzyl)nicotinamide (48.0 mg, 0.100 mmol), 3-(1-fluorocyclopropyl)-1H-1,2,4-triazole (18 mg, 0.140 mmol), copper(I) iodide (21.0 mg, 0.11 mmol), K₃PO₄ (42.5 mg, 0.20 mmol) and (R,R)-(−)-N,N′-dimethyl-1,2-cyclohexanediamine (0.017 mL, 0.11 mmol) in DMF (1 mL) was degassed with N₂ and heated to 120° C. in a sealed tube for 16 h. The cooled mixture was purified by Isolute(Trademark) SCX-2 cartridge and by MDAP to afford the title compound (2 mg). LCMS: mi/480 [M+H]⁺. ¹H NMR (600 MHz, DMSO-d₆) delta ppm 1.21-1.27 (m, 2H) 1.39 (d, J=6.42 Hz, 3H) 1.44-1.53 (m, 2H) 4.31 (d, J=5.69 Hz, 2H) 5.15 (app spt, J=6.60 Hz, 1H) 6.27 (s, 2H) 7.01 (d, J=8.44 Hz, 2H) 7.23 (d, J=8.44 Hz, 2H) 7.34 (br s, 2H) 7.90 (s, 1H) 8.43 (t, J=5.78 Hz, 1H) 8.67 (s, 1H).

### Preparation of 2,6-diamino-5-(1-(difluoromethyl)-1H-pyrazol-4-yl)-N-(2-fluoro-4-(2,2,2-trifluoroethoxy)benzyl)nicotinamide formate (IX)

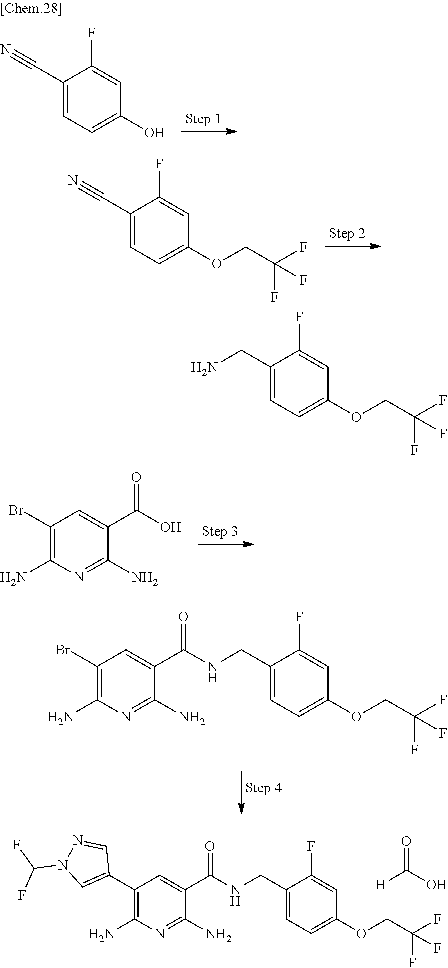

### Step 1: Preparation of 2-fluoro-4-(2,2,2-trifluoroethoxy)benzonitrile

To a stirred suspension of 2-fluoro-4-hydroxybenzonitrile (2.50 g, 18.2 mmol) (CAS 82380-18-5,) and Cs₂COₐ(8.91 g, 27.4 mmol) in DMF (30 mL) at 0° C. was added 2,2,2-trifluoroethyl trifluoromethanesulfonate (3.14 mL, 21.9 mmol). The mixture was allowed to warm to RT and stirred for 16 h. The mixture was diluted with EtOAc (80 mL), washed with water (2×50 mL) and a 5% w/v solution of LiCl (2×50 mL), dried over MgSO₄, filtered and evaporated to afford the title compound (3.93 g). LCMS: m/z 220 [M+H]⁺. ¹H NMR (600 MHz, CHLOROFORM-d) delta ppm 4.41 (q, J=7.83 Hz, 2H) 6.80 (dd, J=10.27, 2.38 Hz, 1H) 6.84 (dd, J=8.62, 2.38 Hz, 1H) 7.60 (m, 1H).

### Step 2: Preparation of (2-fluoro-4-(2,2,2-trifluoroethoxy)phenyl)methanamine hydrochloride

To a stirred solution of 2-fluoro-4-(2,2,2-trifluoroethoxy)benzonitrile (3.92 g, 17.9 mmol) in THF (36 mL) at 0° C. was added BH₃.THF (1 M in THF, 35.8 mL, 35.8 mmol). The mixture was stirred at 0° C. for 30 mins then heated to reflux for 16 h. The mixture was cooled to 0° C. and 2 M HCl (36 mL) was added. The mixture was heated to 100° C. for 2 h, the cooled mixture was evaporated and the residue precipitated from hot EtOAc to afford the title compound (3.49 g). LCMS: m/z 207 [M-NH₂]⁺. ¹H NMR (600 MHz, DMSO-d₆) delta ppm 4.00 (br s, 2H) 4.84 (q, J=8.93 Hz, 2H) 7.00 (dd, J=8.62, 2.38 Hz, 1H) 7.11 (dd, J=11.74, 2.20 Hz, 1H) 7.52 (t, J=8.71 Hz, 1H) 8.27 (br s, 3H).

### Step 3: Preparation of 2,6-diamino-5-bromo-N-(2-fluoro-4-(2,2,2-trifluoroethoxy)benzyl)nicotinamide

To a stirred solution of 2,6-diamino-5-bromonicotinic acid (1.00 g, 4.31 mmol) in DMSO (14 mL) at RT was added Et₃N (2.40 mL, 17.2 mmol), EDC.HCl (0.991 g, 5.17 mmol) and HOBT (0.792 g, 5.17 mmol). The mixture was stirred for 30 mins. (4-(2,2,2-trifluoroethoxy)phenyl)methanamine hydrochloride (5.42 g, 22.4 mmol) was added and the mixture stirred at RT for 16 h then diluted with H₂O (20 mL) and stirred for 2 h, forming a precipitate which was collected by filtration, washed with H₂O (20 mL) and dried under vacuum to afford the title compound (1.33 g). LCMS: m/z 437/439 [M+H]⁺. 1H NMR (600 MHz, DMSO-d6) delta ppm 4.33 (d, J=5.50 Hz, 2H) 4.77 (q, J=8.86 Hz, 2H) 6.31 (br s, 2H) 6.88 (dd, J=8.62, 2.20 Hz, 1H) 6.98 (dd, J=11.83, 2.29 Hz, 1H) 7.07 (br s, 2H) 7.29 (t, J=8.71 Hz, 1H) 8.01 (s, 1H) 8.47 (t, J=5.50 Hz, 1H).

### Step 4: Preparation of 2,6-diamino-5-(1-(difluoromethyl)-1H-pyrazol-4-yl)-N-(2-fluoro-4-(2,2,2-trifluoroethoxy)benzyl)nicotinamide formate

A mixture of 2,6-diamino-5-bromo-N-(2-fluoro-4-(2,2,2-trifluoroethoxy)benzyl)nicotinamide (40 mg, 0.091 mmol), 1-(difluoromethyl)-4-(4,4,5,5-tetramethyl-1,3,2-dioxaborolan-2-yl)-1H-pyrazole (27 mg, 0.11 mmol) (CAS 1206640-82-5), Pd(PPh₃)₄ (5 mg, 4.58 micro mol) in DME (0.92 mL) and 0.5 M aqueous Na₂CO₃ (274 micro L, 0.137 mmol) was degassed with N₂ and heated to 150° C. under microwave (Biotage Initiator) irradiation for 1 h. The cooled mixture was purified by Isolute(Trademark) SCX-2 cartridge and by MDAP to afford the title compound (20 mg). LCMS: m/z 475 [M+H]⁺. ¹H NMR (600 MHz, DMSO-d₆) delta ppm 4.37 (d, J=5.69 Hz, 2H) 4.77 (q, J=8.93 Hz, 2H) 5.97 (br s, 2H) 6.87 (dd, J=8.62, 2.38 Hz, 1H) 6.98 (dd, J=11.92, 2.38 Hz, 1H) 7.04 (br s, 2H) 7.30 (t, J=8.71 Hz, 1H) 7.80 (t, J=59.24 Hz, 1H) 7.86 (s, 1H) 7.98 (s, 1H) 8.13 (s, 1H) 8.31 (s, 1H) 8.43 (br t, J=5.69 Hz, 1H).

### Pharmacological Test Examples

The following pharmacological tests were conducted using the compound I to IX.

Plasmodium falciparum strain 3D7 (chloroquine-sensitive) and K1 (chloroquine-resistant) were obtained from Kitasato University and used for testing antimalarial activities in vitro. The cultivation of P. falciparum was conducted according to Trager's method (Trager, W and Jensen and J., Human malaria parasites in continuous culture. Science, 193:673-677, 1976) with some modification. Precisely, parasites were kept in culture flasks with RPMI1640 medium supplemented with 10% human plasma and 2% fresh human erythrocytes and incubated at 37° C. with the gas condition of 5% CO2 and 5% 02. The parasitemia (percentage of infected erythrocytes to total erythrocytes) were kept within 0.25-10%. Culture medium were replaced and fresh erythrocytes were supplied every 2-3 days. Drug susceptibility test was conducted according to Desjardins's method (Desjardins, R. E., Canfield, C. J., Haynes, D. E. and Chulay, J. D., Quantitative assessment of antimalarial activity in vitro by a semiautomated microdilution technique. Antimicrob. Agents Chemother., 16:710-718, 1979) with some modification. The compounds I to IX and known antimalarial agents (chloroquine and artemisinin) were tested at the same time. Precisely, 199 micro L of parasite cultures (2% hematocrit and 0.75-1% parasitemia) and 1 micro L of compound solution serially diluted in DMSO were poured into every well in 96-well titer plates and final drug concentrations were set within 0.001-1 micro g/mL. The plates were kept at 37° C. with the gas condition of 5% CO2 and 5% O2 for 72 hours and then parasite growth was quantified with Makler's method to detect plasmodial lactate dehydrogenase (Makler, M. T., Rise, J. M., Williams, J. A., Bancroft, J. E., Piper, R. C., Gibbins, B. L. and Hinrichs, D. J., Parasite lactate dehydrogenase as an Assay for Plasmodium falciparum drug sensitivity. Am. J. Med. Hyg., 48:739-741, 1993) with some modification. Precisely, culture plate was kept in freezer overnight and then thawed at 37° C. to disrupt the erythrocytes and parasite cells. In the new 96-well titer plates, 100 micro L of enzyme reaction solution (110 mM Li-lactate, 0.5 mM acetylpyridine-adenine dinucleotide, 50 mM Tris (pH 7.5), 10 mM EDTA, 50 mM KCl and 15 g/L PEG6000) and 20 micro L of freeze-thaw culture were mixed in each well and then kept at room temperature for 30 minutes. The detection solution was prepared by mixing equal volume of 2 mg/mL nitro blue tetrazolium and 0.1 mg/mL phenazine ethosulfate and 20 micro L of the solution was added to each well. After the incubation at room temperature in the dark for 90 minutes, absorbance at 660 nm was analysed and IC50s were calculated from dose response curve. The IC50 values of the compounds I to IX are reported in Table 1.

TABLE 1

| | Pf_3D7 | | Pf_K1 | |
|---|---|---|---|---|
| compound | IC50 [μg/mL] | IC50 [μM] | IC50 [μg/mL] | IC50 [μM] |
| I | 0.026 | 0 053 | 0.043 | 0.030 |
| II | 0.020 | 0.042 | 0.025 | 0.053 |
| III | 0.078 | 0.162 | 0.121 | 0.249 |
| IV | 0.000 | 0.125 | 0.031 | 0.171 |
| V | 0.047 | 0.099 | 0.075 | 0.160 |
| VI | 0.024 | 0.048 | 0.038 | 0.075 |
| VII | 0.016 | 0.030 | 0.021 | 0.041 |
| VIII | 0.020 | 0.054 | 0.031 | 0.064 |
| IX | 0.050 | 0.113 | 0.083 | 0.160 |

The following pharmacological tests in the humanized murine malaria model were conducted using the compounds I, II and V at The Art of Discovery S.L. (Bizkaia, Spain). Briefly, mice engrafted with human erythrocytes were intravenously infected with parasitized red blood cells 72 h before drug treatment inception. The effect of treatment on parasitemia was assessed by measuring the percentage of infected erythrocytes in peripheral blood.

Plasmodium falciparum Pf3D70087/N9, the parasite strain used in this experiment, was described in reference (1). Immunodeficient female NSG (NOD-scid IL-2Rγnull) mice were engrafted with human erythrocytes to have a minimum of 40% of human erythrocytes circulating in peripheral blood during the whole experiment. Each mouse was intraperitoneally (i.p.) or intravenously (i.v.) inoculated with 1 mL (i.p.) or 0.7 mL (i.v.) of 50%-75% hematocrit erythrocyte suspension in RPMI1640 medium, 25% (vol/vol) decomplemented human serum, 3.1 mM hypoxanthine. Then humanized NSG mice were infected with peripheral blood from donor mice by intravenous injection of 0.3 mL of 1.17×10⁸ parasitized-erythrocytes per ml suspension. Drug treatment was started after 72-h post infection. In the treatment, 10 ml/kg of compound solution were orally administered to each mouse, once daily for three or four days (three days for compounds I and V, four days for compound II, respectively). An analysis of the parasite population in peripheral blood was performed by flow cytometry as shown in reference (2) and the outcome was shown as the parasitemia expressed as the % of parasitized erythrocytes to the total erythrocytes in peripheral blood. Briefly, serial 2 micro L blood samples of peripheral blood from infected mice were stained with TER-119-Phycoerythrine (marker of murine erythrocytes) and SYTO-16 (nucleic acid dye) and then analyzed by flow cytometry. The therapeutic efficacies were expressed as ED90, the daily doses that reduce by 90% parasitemia to untreated control animals after seven days post infection. The obtained ED90s of compounds I, II and V were 3.2 mg/kg/day, 5.0 mg/kg/day and 6.2 mg/kg/day respectively.

(1) Angulo-Barturen et al. A murine model of falciparum-malaria by in vivo selection of competent strains in nonmyelodepleted mice engrafted with human erythrocytes. Plos One. 2008 May 21; 3(5):e2252.
(2) Jimenez-Diaz et al. Quantitative measurement of Plasmodium-infected erythrocytes in murine models of malaria by flow cytometry using bidimensional assessment of SYTO-16 fluorescence. Cytometry A. 2009 March, 75(3):225-35.

Regarding the prophylactic effect, two possibilities are expected. Firstly, inhibition of parasite maturation at liver stage in which the sporozoites injected by mosquito develop to mature schizonts inside hepatocytes. It was reported that the one of major GPI-anchored proteins, MSP-1, has an essential role for this maturation [1], so Gwt1p-inhibitors are expected to be effective not only in blood stage but also liver stage. To confirm this, the ex vivo hepatocyte infection using sporozoites of rodent malaria parasites produced in mosquito [2, 3] can be conducted. Secondly, inhibition of parasite growth at the first cycle of blood stage can eliminate parasites before the appearance of malaria symptoms. The therapeutic activity in blood stage is confirmed in the murine model with rodent malaria parasite [3]. The same experiment with pretreatment by Gwt1p-inhibitors can be conducted to confirm the prophylactic effect in the blood stage.

(1) Kawabata Y, Udono H, Honma K et al. Merozoite Surface Protein 1-Specific Immune Response is Protective against Exoerythrocytic Forms of Plasmodium yoelii. Infection and Immunity 2002; 70:6075-82.

(2) Hovlid M L, Winzeler E A. Phenotypic screens in antimalarial drug discovery. Trends Parasitol. 2016:32:697-707.

(3) Kato N, Comer E, Sakata-Kato T et al. Diversity-oriented synthesis yields novel multistage antimalarial inhibitors. Nature 2016; 538:344-349.

1. A compound selected from the group consisting of:
(R)-2,6-diamino-5-(3-(trifluoromethyl)-1H-1,2,4-triazol-1-yl)-N-(4-((1,1,1-trifluoropropan-2-yl)oxy)benzyl)nicotinamide represented by the following formula (I): 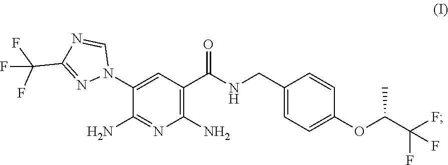 [SMILES: C[C@@H](Oc1ccc(CNC(=O)c2cc(-n3cnc(C(F)(F)F)n3)c(N)nc2N)cc1)C(F)(F)F]
(R)-2,6-diamino-5-(1-(difluoromethyl)-1H-pyrazol-4-yl)-N-(4-((1,1,1-trifluoropropan-2-yl)oxy)benzyl)nicotinamide represented by the following formula (II): 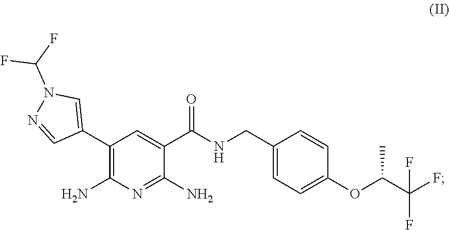 [SMILES: C[C@@H](Oc1ccc(CNC(=O)c2cc(-c3cnn(C(F)F)c3)c(N)nc2N)cc1)C(F)(F)F]
(R)-2,6-diamino-5-(1-(difluoromethyl)-3-methyl-1H-pyrazol-4-yl)-N-(4-((1,1,1-trifluoropropan-2-yl)oxy)benzyl)nicotinamide represented by the following formula (III): 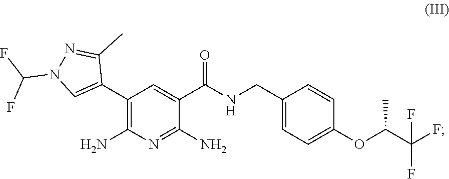 [SMILES: Cc1nn(C(F)F)cc1-c1cc(C(=O)NCc2ccc(O[C@H](C)C(F)(F)F)cc2)c(N)nc1N]
2,6-diamino-N-(4-(2,2,2-trifluoroethoxy)benzyl)-5-(3-(trifluoromethyl)-1H-1,2,4-triazol-1-yl)nicotinamide represented by the following formula (IV): 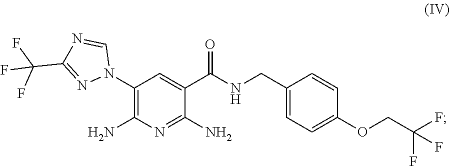 [SMILES: Nc1nc(N)c(-n2cnc(C(F)(F)F)n2)cc1C(=O)NCc1ccc(OCC(F)(F)F)cc1]
(R)-2,6-diamino-5-(3-(difluoromethyl)-1H-1,2,4-triazol-1-yl)-N-(4-((1,1,1-trifluoropropan-2-yl)oxy)benzyl)nicotinamide represented by the following formula (V): 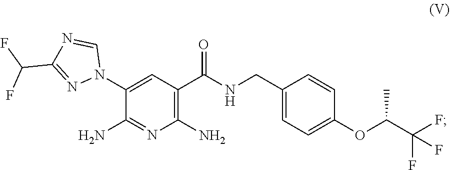 [SMILES: C[C@@H](Oc1ccc(CNC(=O)c2cc(-n3cnc(C(F)F)n3)c(N)nc2N)cc1)C(F)(F)F]
(R)-2,6-diamino-5-(3-(2,2,2-trifluoroethyl)-1H-1,2,4-triazol-1-yl)-N-(4-((1,1,1-trifluoropropan-2-yl)oxy)benzyl)nicotinamide represented by the following formula (VI): 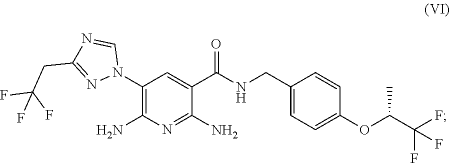 [SMILES: C[C@@H](Oc1ccc(CNC(=O)c2cc(-n3cnc(CC(F)(F)F)n3)c(N)nc2N)cc1)C(F)(F)F]
2,6-diamino-N-(4-(2,2,2-trifluoroethoxy)benzyl)-5-(3-(1-(trifluoromethyl)cyclopropyl)-1H-1,2,4-triazol-1-yl)nicotinamide represented by the following formula (VII): 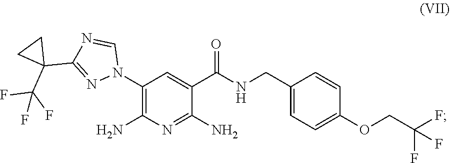 [SMILES: Nc1nc(N)c(-n2cnc(C3(C(F)(F)F)CC3)n2)cc1C(=O)NCc1ccc(OCC(F)(F)F)cc1]
(R)-2,6-diamino-5-(3-(1-fluorocyclopropyl)-1H-1,2,4-triazol-1-yl)-N-(4-((1,1,1-trifluoropropan-2-yl)oxy)benzyl)nicotinamide represented by the following formula (VIII): 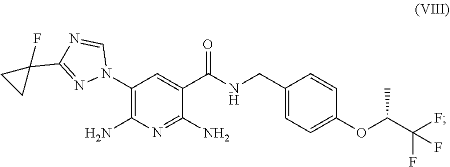 [SMILES: C[C@@H](Oc1ccc(CNC(=O)c2cc(-n3cnc(C4(F)CC4)n3)c(N)nc2N)cc1)C(F)(F)F]
and
2,6-diamino-5-(1-(difluoromethyl)-1H-pyrazol-4-yl)-N-(2-fluoro-4-(2,2,2-trifluoroethoxy)benzyl)nicotinamide represented by the following formula (IX): 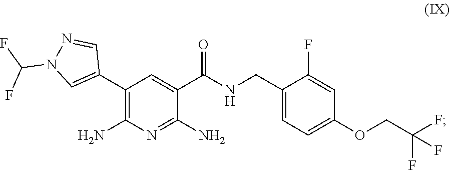 [SMILES: Nc1nc(N)c(-c2cnn(C(F)F)c2)cc1C(=O)NCc1ccc(OCC(F)(F)F)cc1F]
or a pharmaceutically acceptable salt thereof.

2. (R)-2,6-Diamino-5-(3-(trifluoromethyl)-1H-1,2,4-triazol-1-yl)-N-(4-((1,1,1-trifluoropropan-2-yl)oxy)benzyl)nicotinamide represented by the following formula (I): 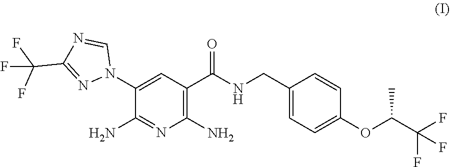 [SMILES: C[C@@H](Oc1ccc(CNC(=O)c2cc(-n3cnc(C(F)(F)F)n3)c(N)nc2N)cc1)C(F)(F)F]
or a pharmaceutically acceptable salt thereof.

3. (R)-2,6-Diamino-5-(1-(difluoromethyl)-1H-pyrazol-4-yl)-N-(4-((1,1,1-trifluoropropan-2-yl)oxy)benzyl)nicotinamide represented by the following formula (II): 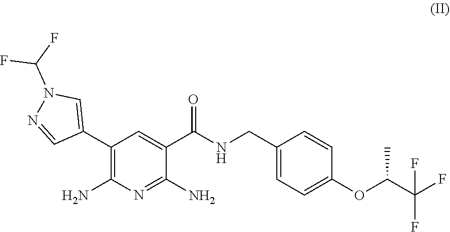 [SMILES: C[C@@H](Oc1ccc(CNC(=O)c2cc(-c3cnn(C(F)F)c3)c(N)nc2N)cc1)C(F)(F)F]
or a pharmaceutically acceptable salt thereof.

4. (R)-2,6-Diamino-5-(1-(difluoromethyl)-3-methyl-1H-pyrazol-4-yl)-N-(4-((1,1,1-trifluoropropan-2-yl)oxy)benzyl)nicotinamide represented by the following formula (III): 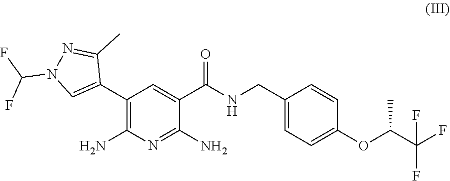 [SMILES: Cc1nn(C(F)F)cc1-c1cc(C(=O)NCc2ccc(O[C@H](C)C(F)(F)F)cc2)c(N)nc1N]
or a pharmaceutically acceptable salt thereof.

5. 2,6-Diamino-N-(4-(2,2,2-trifluoroethoxy)benzyl)-5-(3-(trifluoromethyl)-1H-1,2,4-triazol-1-yl)nicotinamide represented by the following formula (IV): 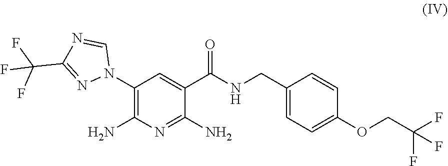 [SMILES: Nc1nc(N)c(-n2cnc(C(F)(F)F)n2)cc1C(=O)NCc1ccc(OCC(F)(F)F)cc1]
or a pharmaceutically acceptable salt thereof.

6. (R)-2,6-Diamino-5-(3-(difluoromethyl)-1H-1,2,4-triazol-1-yl)-N-(4-((1,1,1-trifluoropropan-2-yl)oxy)benzyl)nicotinamide represented by the following formula (V): 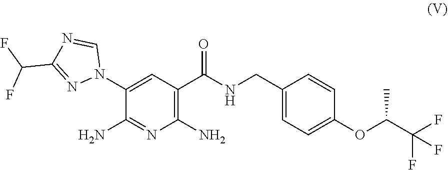 [SMILES: C[C@@H](Oc1ccc(CNC(=O)c2cc(-n3cnc(C(F)F)n3)c(N)nc2N)cc1)C(F)(F)F]
or a pharmaceutically acceptable salt thereof.

7. (R)-2,6-Diamino-5-(3-(2,2,2-trifluoroethyl)-1H-1,2,4-triazol-1-yl)-N-(4-((1,1,1-trifluoropropan-2-yl)oxy)benzyl)nicotinamide represented by the following formula (VI): 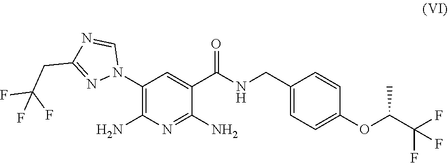 [SMILES: C[C@@H](Oc1ccc(CNC(=O)c2cc(-n3cnc(CC(F)(F)F)n3)c(N)nc2N)cc1)C(F)(F)F]
or a pharmaceutically acceptable salt thereof.

8. 2,6-Diamino-N-(4-(2,2,2-trifluoroethoxy)benzyl)-5-(3-(1-(trifluoromethyl)cyclopropyl)-1H-1,2,4-triazol-1-yl)nicotinamide represented by the following formula (VII): 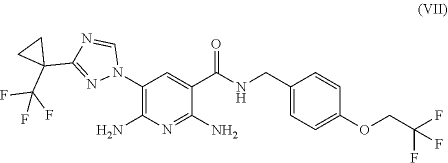 [SMILES: Nc1nc(N)c(-n2cnc(C3(C(F)(F)F)CC3)n2)cc1C(=O)NCc1ccc(OCC(F)(F)F)cc1]
or a pharmaceutically acceptable salt thereof.

9. (R)-2,6-Diamino-5-(3-(1-fluorocyclopropyl)-1H-1,2,4-triazol-1-yl)-N-(4-((1,1,1-trifluoropropan-2-yl)oxy)benzyl)nicotinamide represented by the following formula (VIII): 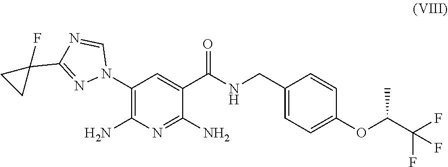 [SMILES: C[C@@H](Oc1ccc(CNC(=O)c2cc(-n3cnc(C4(F)CC4)n3)c(N)nc2N)cc1)C(F)(F)F]
or a pharmaceutically acceptable salt thereof.

10. 2,6-Diamino-5-(1-(difluoromethyl)-1H-pyrazol-4-yl)-N-(2-fluoro-4-(2,2,2-trifluoroethoxy)benzyl)nicotinamide represented by the following formula (IX): 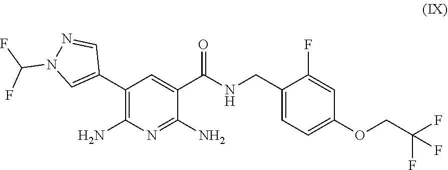 [SMILES: Nc1nc(N)c(-c2cnn(C(F)F)c2)cc1C(=O)NCc1ccc(OCC(F)(F)F)cc1F]
or a pharmaceutically acceptable salt thereof.

11. A pharmaceutical composition comprising the compound or pharmaceutically acceptable salt thereof according to claim 1.

12. An antimalarial agent comprising the compound or pharmaceutically acceptable salt thereof according to claim 1.

13. A malaria prophylactic agent comprising the compound or pharmaceutically acceptable salt thereof according to claim 1.

14. A method of treating or preventing malaria, comprising administering to a subject the compound or pharmaceutically acceptable salt thereof according to claim 1.

15.-17. (canceled)
